(19) United States
(12) Patent Application Publication
(10) Pub. No.: US 2022/0105207 A1
(43) Pub. Date: Apr. 7, 2022

(54) Macrocyclic Agents for Targeted Dual-Modality PET and MRI Imaging of Cancer

(71) Applicant: Wisconsin Alumni Research Foundation, Madison, WI (US)

(72) Inventors: Jamey Weichert, Sun Prairie, WI (US); Reinier Hernandez, Madison, WI (US); Ray Zhang, Madison, WI (US); Anatoly Pinchuk, Fitchburg, WI (US)

(21) Appl. No.: 17/552,984

(22) Filed: Dec. 16, 2021

Related U.S. Application Data
(63) Continuation of application No. 16/851,585, filed on Apr. 17, 2020, now abandoned.
Application No. 16/851,585 is a continuation of application No. 16/222,364, filed on Dec. 17, 2018, now abandoned.
(60) Provisional application No. 62/599,169, filed on Dec. 15, 2017.

(51) Int. Cl. A61K 51/04; A61K 9/00; A61B 5/055; C07B 59/00; A61K 49/00; C07F 9/6524; A61K 49/10; A61K 49/08

(52) U.S. Cl. CPC A61K 51/0474; A61K 9/0019; A61B 5/055; C07B 59/002; A61K 51/0497; A61K 51/0408; C07F 9/6524; A61K 49/10; A61K 49/085; A61K 49/0002

(57) ABSTRACT

Dual-modality contrast agents are disclosed herein, having the general formula:

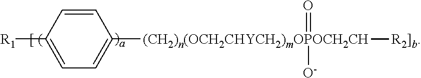

R₁ includes a chelating moiety that is chelated to a Mn²⁺ isotope. The disclosed contrast agents differentially target a wide range of malignant tumor tissues, and can be simultaneously used as contrast agents for both magnetic resonance imaging (MRI) and positron emission topography (PET) imaging. Accordingly, the disclosed contrast agent can be used in diagnosing and monitoring solid tumor cancers.

### CROSS-REFERENCE TO RELATED APPLICATIONS

This application is a continuation of U.S. application Ser. No. 16/222,364 filed on Dec. 17, 2018, which claims the benefit of U.S. provisional Application No. 62/599,169 filed on Dec. 15, 2017. Each of these applications is incorporated by reference herein.

### STATEMENT REGARDING FEDERALLY SPONSORED RESEARCH OR DEVELOPMENT

Not applicable.

### FIELD OF THE DISCLOSURE

This disclosure relates generally to the medical diagnosis/imaging of cancers. In particular, this disclosure directed to (a) manganese-chelating alkylphosphocholine analogs, and (b) methods of using such analogs as dual-modality contrast agents for detecting/imaging cancer tumor cells using both magnetic resonance imaging (MRI) and positron emission tomography (PET) imaging.

### BACKGROUND

Magnetic resonance imaging (MRI) has become an indispensable tool for the diagnosis of a plethora of diseases, ranging from cardiovascular diseases to cancer. Particularly, contrast-enhanced MRI (CEMRI) using contrast agents that modify the T1 and/or T2 relaxation times of water protons within different diseased tissue or process has been proven useful for both anatomical and functional imaging.

Within the context of cancer, most MRI contrast agents rely on differences in vascular parameters, such as perfusion and blood pooling, to achieve tumor-to-normal tissue contrast. While providing useful information, such agents often lack the necessary specificity to provide a clear diagnosis and to effectively monitor responses to targeted therapies. Despite the pressing need for finding new classes of MRI agents with increased tumor-specificity, this endeavor has been hindered by a fundamental disconnection between the saturable nature of most tumor-targeting strategies and the “large” concentration of contrast agent needed to attain an adequate MRI signal. Hence, finding a cancer-specific molecular mechanism that is non-saturable and by which significant MRI contrast agent payloads can be delivered to the tumor is necessary in order to advance cancer-specific MRI-based diagnosis.

We have previously shown that certain compounds having an alkylphosphocholine (APC) backbone display selective and persistent accumulation in a wide variety of malignancies, while showing marginal uptake and rapid clearance from normal tissue. For example, in U.S. Patent Publication No. 2014/0030187, which is incorporated by reference herein in its entirety, Weichert et al. disclose using analogs of the base compound 18-(p-iodophenyl)octadecyl phosphocholine (NM404; see FIG. 1) for detecting and locating, as well as for treating, a wide variety of solid tumor cancers. If the iodo moiety is an imaging-optimized radionuclide, such as iodine-124 ([¹²⁴I]-NM404), the analog can be used in positron emission tomography-computed tomography (PET/CT) or single-photon emission computed tomography (SPECT) imaging of solid tumors. Alternatively, if the iodo moiety is a radionuclide optimized for delivering therapeutic doses of radiation to the solid tumors cells in which the analog is taken up, such as iodine-125 or iodine-131 ([¹²⁵I]-NM404 or [¹³¹I]-NM404), the analog can be used to treat solid tumors.

It has been demonstrated that the tumor uptake of APCs is unaffected by the mass dose administered to a subject. Moreover, extensive structure-activity relation studies revealed that significant modification can be made to the aryl end of the molecule, while retaining tumor uptake and specificity, suggesting the possibility of developing APC analogues featuring MRI-reportable moieties.

Historically, coordination compounds of Gadolinium (III) (Gd³⁺) have been the primary contrast agents used in contrast-enhanced MRI, with several open and macrocyclic chelates of Gd³⁺ being routinely used in clinical practice. In U.S. Patent Publication No. 2017/0128572, which is incorporated by reference herein in its entirety, Weichert et al. disclose a range of gadolinium (Gd) chelates having the tumor-targeting APC backbone that can be used as long-lived tumor-specific MRI contrast agents and as neutron capture therapy agents.

In spite of the favorable properties of Gd³⁺ for use in MRI contrast agents, namely high nuclear spin (7/2) which results in elevated relativities (r1), significant safety concerns about the use Gd-containing contrast agents have been raised, particularly in patients with impaired renal function. Recently, evidence of Gd³⁺ deposition within deep regions of the brain after repeated administration of Gd-based contrasts agents have resulted in a ban for the use of non-macrocyclic (linear) Gd compounds as MRI contrast agents in Europe. Accordingly, it is likely that, going forward, only macrocyclic chelates will be used clinically as Gd-based MRI contrast agents.

An alternative to using Gd-based contrast agents is to use compounds containing Manganese II (Mn²⁺) as MRI contrast agents (see, e.g., Pan et al., Tetrahedron, 2011 Nov. 4; 67(44): 8431-8444). The use of Mn as an alternative to Gd affords superior nuclear and magnetic properties, and is also appealing in terms of safety and long term deposition. Furthermore, unlike Gd, Mn can occur as a positron emitting paired isotope (Mn-51 or Mn-52). Additionally, the coordination chemistry of Mn²⁺, which has been extensively described, resembles that of Gd³⁺, and a wide variety of linear and macrocyclic chelates bind to Mn²⁺ with excellent thermodynamic and kinetic stability.

However, there have been reported toxicity and clinical performance issues with some proposed Mn-based MRI contrast agents. In addition, none of the previously disclosed Mn-based chelates exhibit the desirable tumor-targeting characteristics of our previously-disclosed Gd/APC-based MRI contrast agents.

Accordingly, there is a need in the art for improved MRI contrast agents that are not Gd-based and that target and are preferentially retained by cancerous tumor tissues. Furthermore, given the recent development of simultaneous hybrid PET/MRI scanners, it would be desirable if the improved MRI contrast agents were also capable of functioning as PET contrast agents.

### BRIEF SUMMARY

We disclose herein Mn-chelates that include a cancerous tumor-targeting APC backbone that are highly stable, relatively hydrophilic, and, unlike other Mn-chelates, cleared by the liver rather than by the kidneys.

Due to the selective and elevated tumor uptake of the APCs, excellent tumor to background ratios can be attained using a fraction of the mass dose typically needed for CEMRI, which significantly reduces the risk of toxicity. Furthermore, the prolonged retention of the contrast agent within tumor cell will allow for the unequivocal discrimination between cancer cells and other radiological processes such as radiation necrosis or inflammation, which are often misdiagnosed using current Gd-based compounds. In addition, the disclosed contrast agents would enable the detection of disseminated metastasis and lymph node invasion that present delayed contrast uptake and are not detected by Gd-MRI.

Furthermore, the use of positron-emitting isotopes of Mn (Mn-52 and Mn-51) in the disclosed contrast agents provides the first true dual-modality positron emission tomography (PET/MRI) contrast agent which, for the first time, would marry the superb spatial resolution of MRI with the excellent detection sensitivity and quantitative character of PET, thus closing the existing resolution gap between the imaging agents and the scanners in these two powerful imaging modalities. This PET/MRI approach would be of interest to the radiology and nuclear medicine communities, given the recent availability of simultaneous hybrid PET/MRI scanners.

Accordingly, in a first aspect, this disclosure encompasses a dual-modality contrast agent that can be used in both MRI and PET imaging of cancer. The dual-modality contrast agent is a phospholipid metal chelate compound having the general formula:

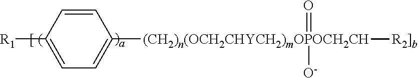

or a salt thereof. R₁ comprises a chelating moiety that is chelated to a Mn²⁺ isotope, a is 0 or 1; n is an integer from 12 to 30; m is 0 or 1; Y is —H, —OH, —COOH, —COOX, —OCOX, or —OX, wherein X is an alkyl or an aryl; R₂ is —N⁺H₃, —N⁺H₂Z, —N⁺HZ₂, or —N⁺Z₃, wherein each Z is independently an alkyl or an aroalkyl; and b is 1 or 2. Examples of Mn²⁺ isotopes that could be used include Mn²⁺-52 and Mn²⁺-51.

In some embodiments, the chelating moiety is 1,4,7,10-tetraazacyclododecane-1,4,7-triacetic acid (DO3A) or one of its derivatives; 1,4,7-triazacyclononane-1,4-diacetic acid (NODA) or one of its derivatives; 1,4,7-triazacyclononane-1,4,7-triacetic acid (NOTA) or one of its derivatives; 1,4,7,10-tetraazacyclododecane-1,4,7,10-tetraacetic acid (DOTA) or one of its derivatives; 1,4,7-triazacyclononane,1-glutaric acid-4,7-diacetic acid (NODAGA) or one of its derivatives; 1,4,7,10-tetraazacyclodecane,1-glutaric acid-4,7,10-triacetic acid (DOTAGA) or one of its derivatives; 1,4,8,11-tetraazacyclotetradecane-1,4,8,11-tetraacetic acid (IETA) or one of its derivatives; 1,4,8,11-tetraazabicyclo[6.6.2]hexadecane-4,11-diacetic acid (CB-TE2A) or one of its derivatives; diethylene triamine pentaacetic acid (DTPA), its diester, or one of its derivatives; 2-cyclohexyl diethylene triamine pentaacetic acid (CHX-A″-DTPA) or one of its derivatives; deforoxamine (DFO) or one of its derivatives; 1,2-[[6-carboxypyridin-2-yl]methylamino]ethane (H₂dedpa) or one of its derivatives; and DADA or one of its derivatives, wherein DADA comprises the structure:

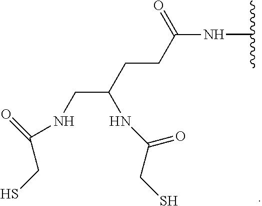 [SMILES: *NC(=O)CCC(CNC(=O)CS)NC(=O)CS]

In some embodiments, the chelating moiety that is chelated to the Mn²⁺ isotope is:

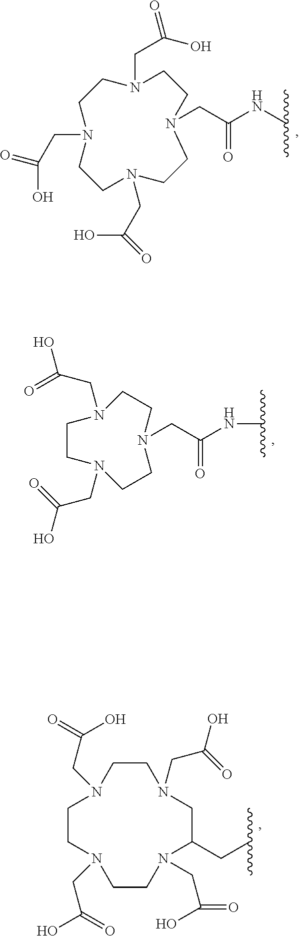 [SMILES: *NC(=O)CN1CCN(CC(=O)O)CCN(CC(=O)O)CCN(CC(=O)O)CC1 ; *NC(=O)CN1CCN(CC(=O)O)CCN(CC(=O)O)CC1 ; *CC1CN(CC(=O)O)CCN(CC(=O)O)CCN(CC(=O)O)CCN1CC(=O)O]

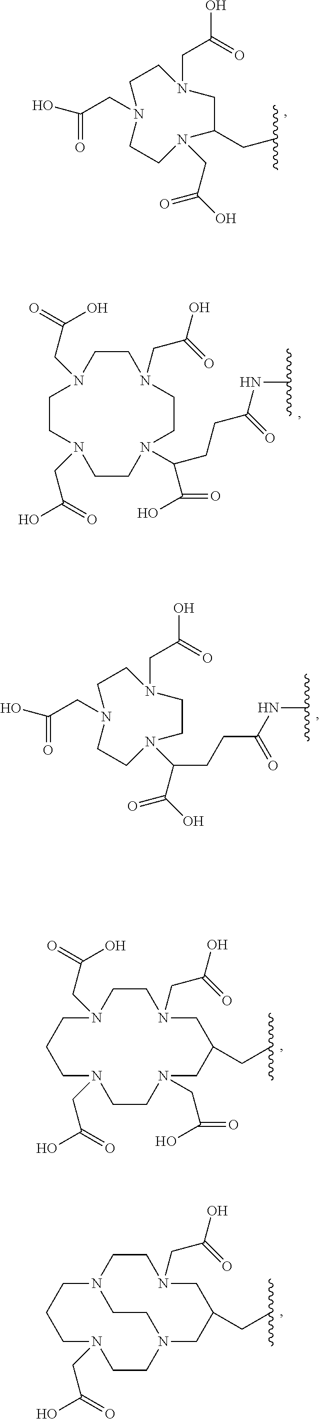 [SMILES: *CC1CN(CC(=O)O)CCN(CC(=O)O)CCN1CC(=O)O ; *NC(=O)CCC(C(=O)O)N1CCN(CC(=O)O)CCN(CC(=O)O)CCN(CC(=O)O)CC1 ; *NC(=O)CCC(C(=O)O)N1CCN(CC(=O)O)CCN(CC(=O)O)CC1 ; *CC1CN(CC(=O)O)CCN(CC(=O)O)CCCN(CC(=O)O)CCN(CC(=O)O)C1 ; *CC1CN2CCN(CCCN(CC(=O)O)CC2)CCN(CC(=O)O)C1]

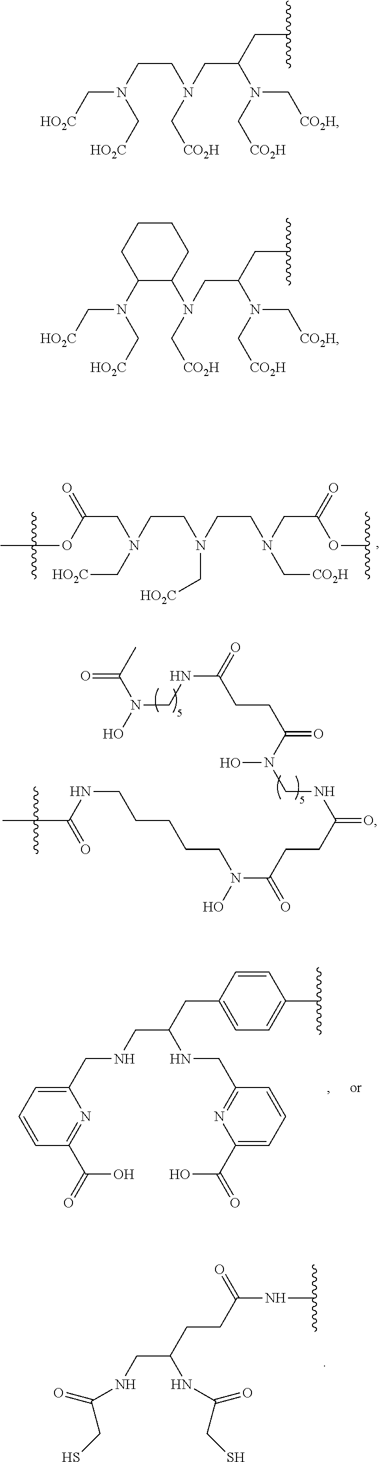

In some embodiments, the chelating moiety chelated to the Mn²⁺ isotope is:

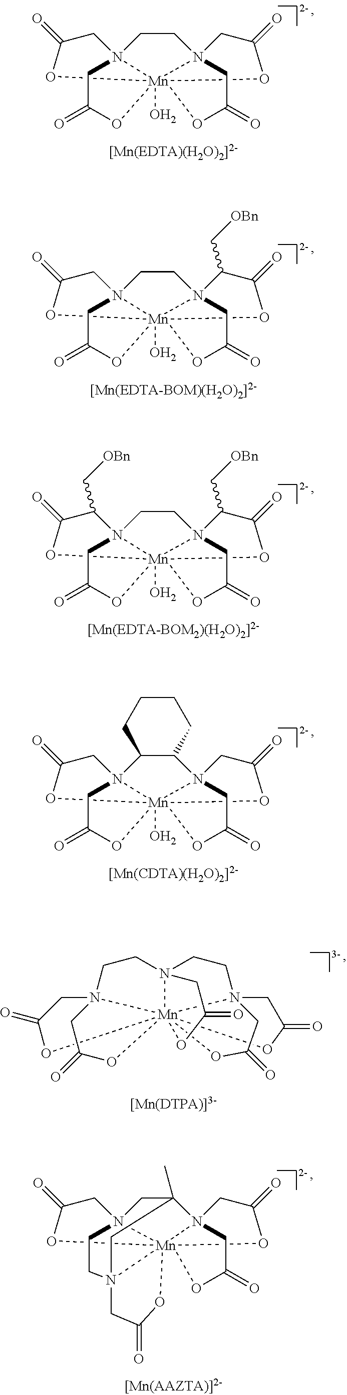

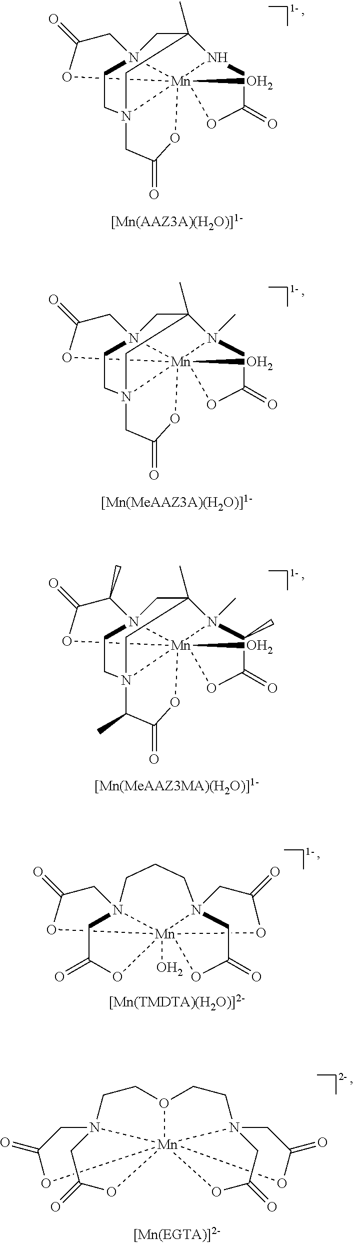

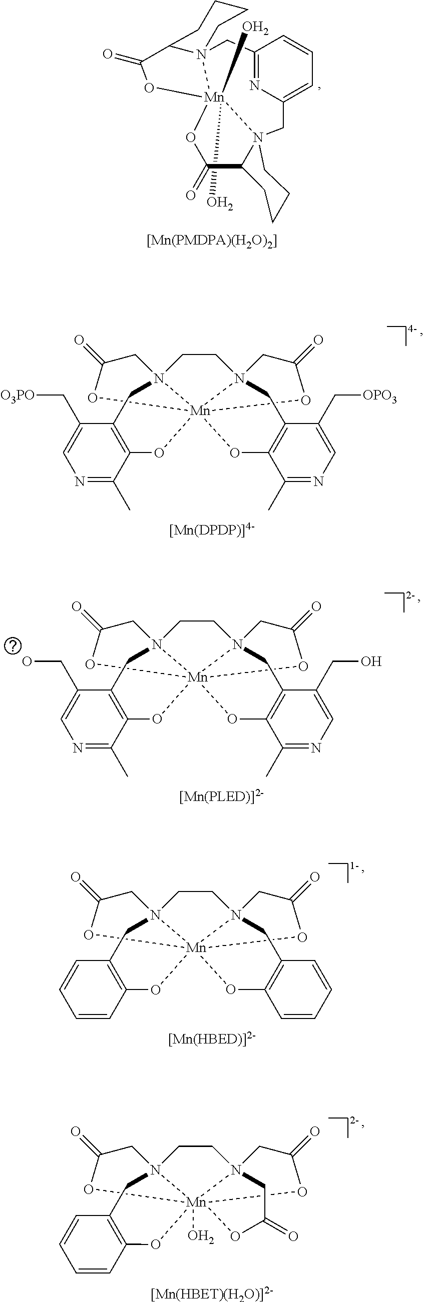

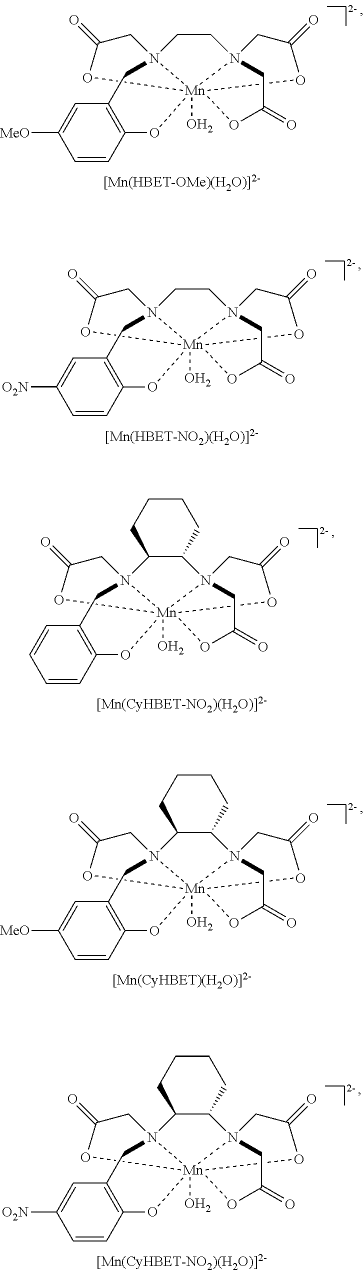

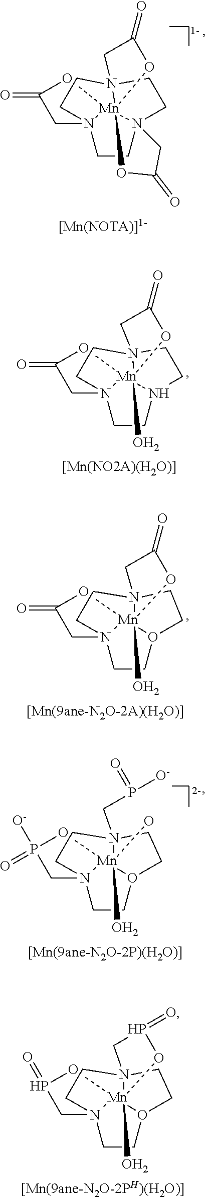

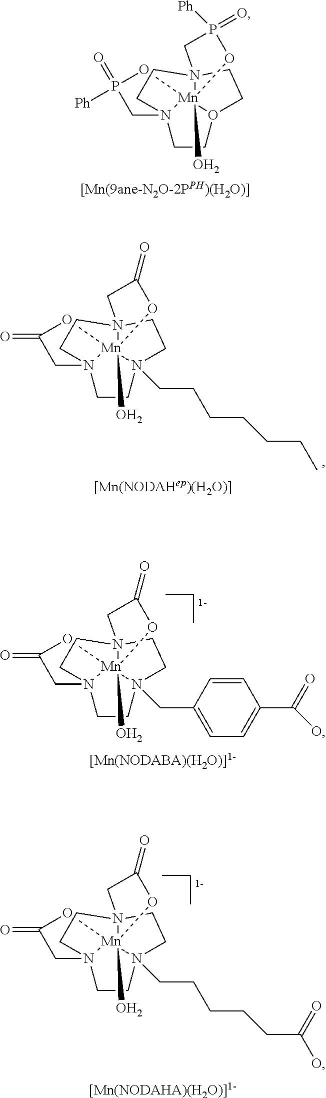

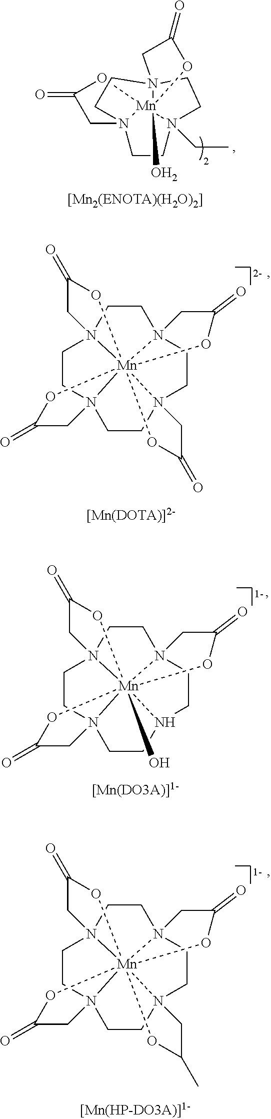

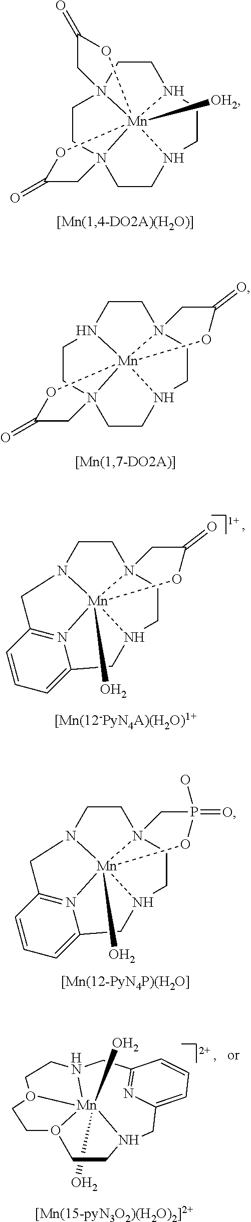

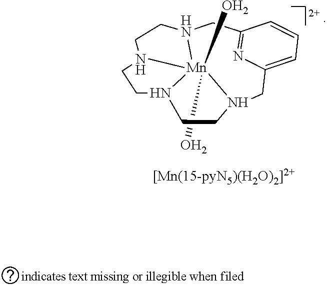

In some embodiments, the chelating moiety that is chelated to the Mn²⁺ isotope is a macrocycle.

In some embodiments, a is 1 (aliphatic aryl-alkyl chain). In other embodiments, a is 0 (aliphatic alkyl chain).

In some embodiments, m is 1 (acylphospholipid series). In some such embodiments, n is an integer between 12 and 20. In some embodiments, Y is —OCOX, —COOX or —OX.

In some embodiments, X is —CH₂CH₃ or —CH₃.

In some embodiments, m is 0 (alkylphospholipid series).

In some embodiments, b is 1.

In some embodiments, n is 18.

In some embodiments, R₂ is —N⁺Z₃. In some such embodiments, each Z is independently —CH₂CH₃ or —CH₃. In some such embodiments, each Z is —CH₃.

In some embodiments, the contrast agent has the chemical structure:

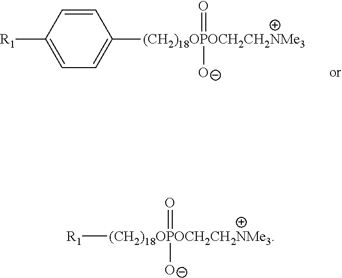

In some embodiments, the contrast agent, excluding the chelated Mn²⁺ isotope, has the chemical structure:

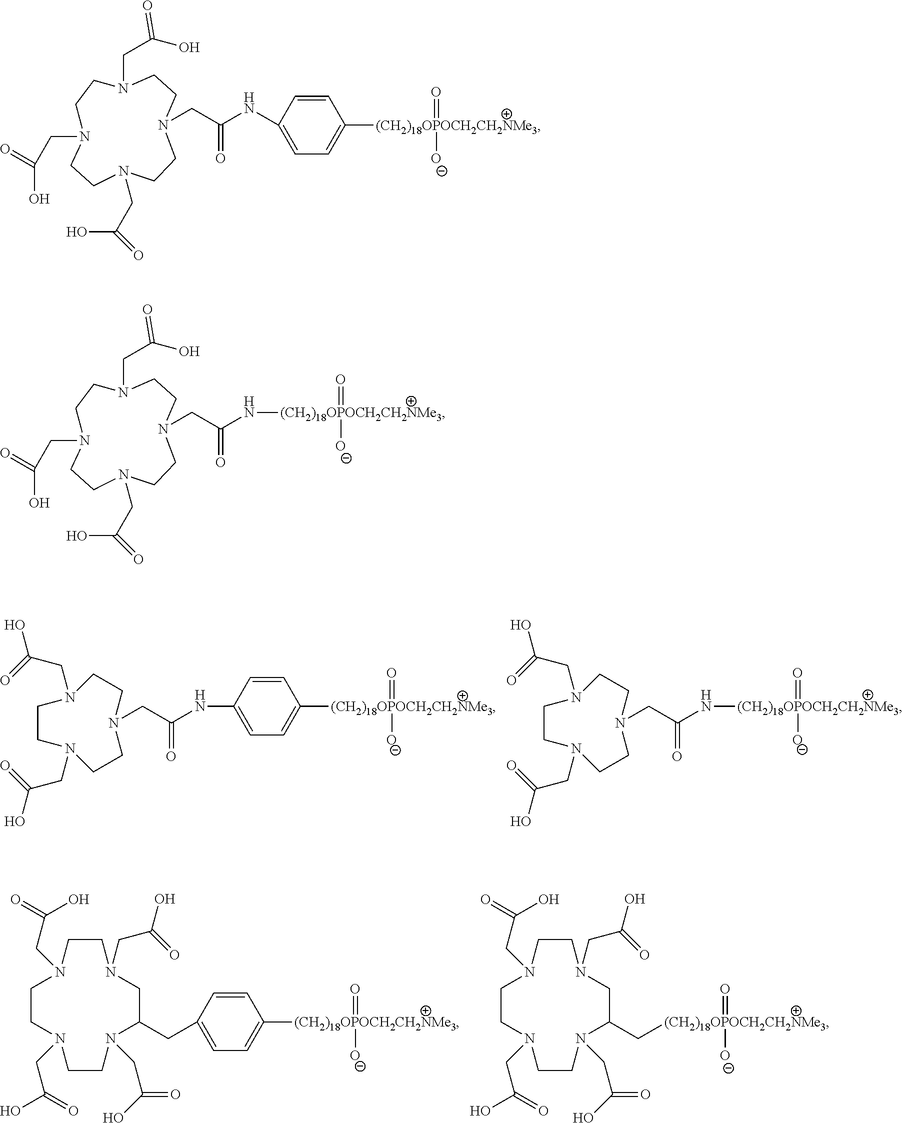

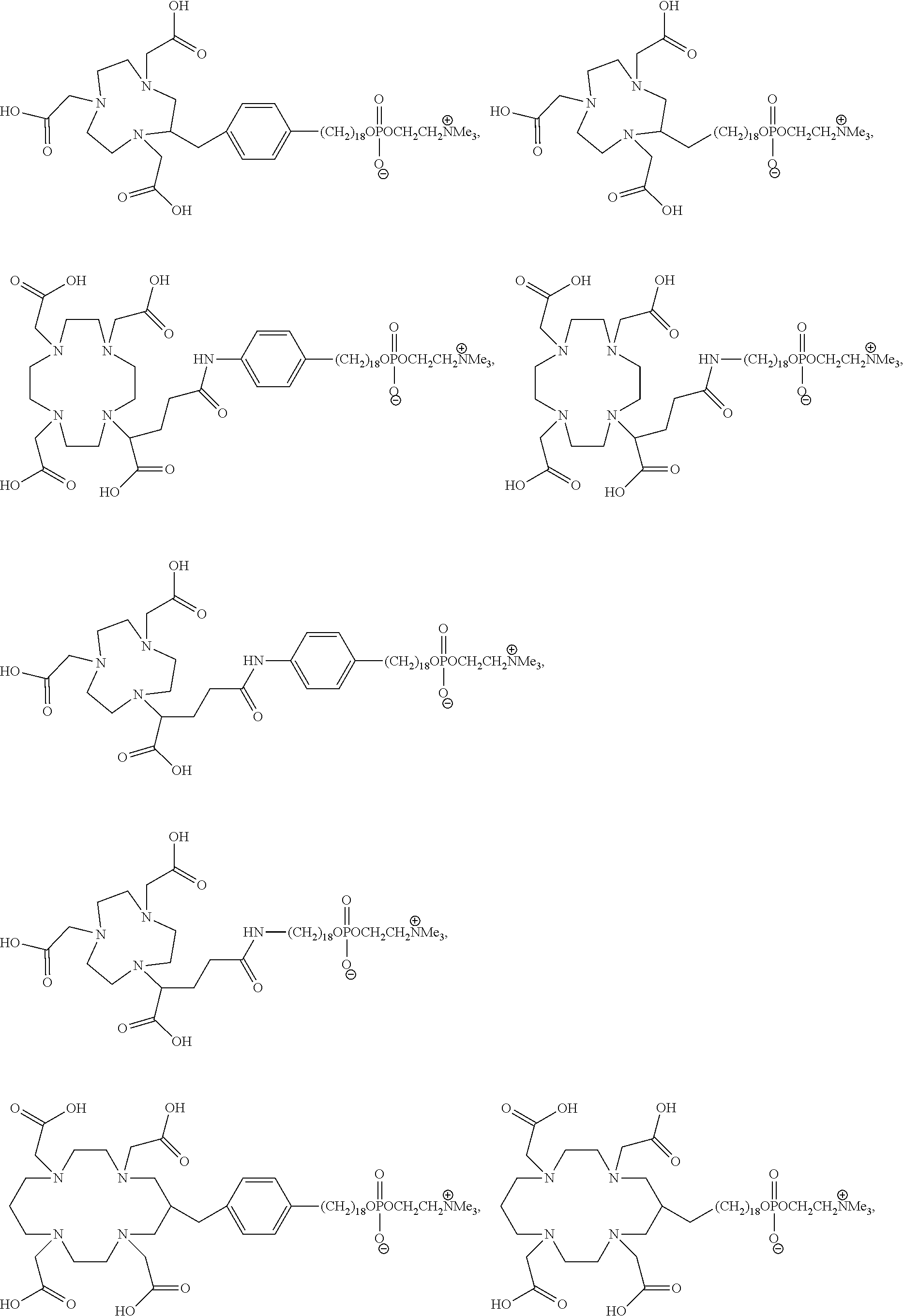

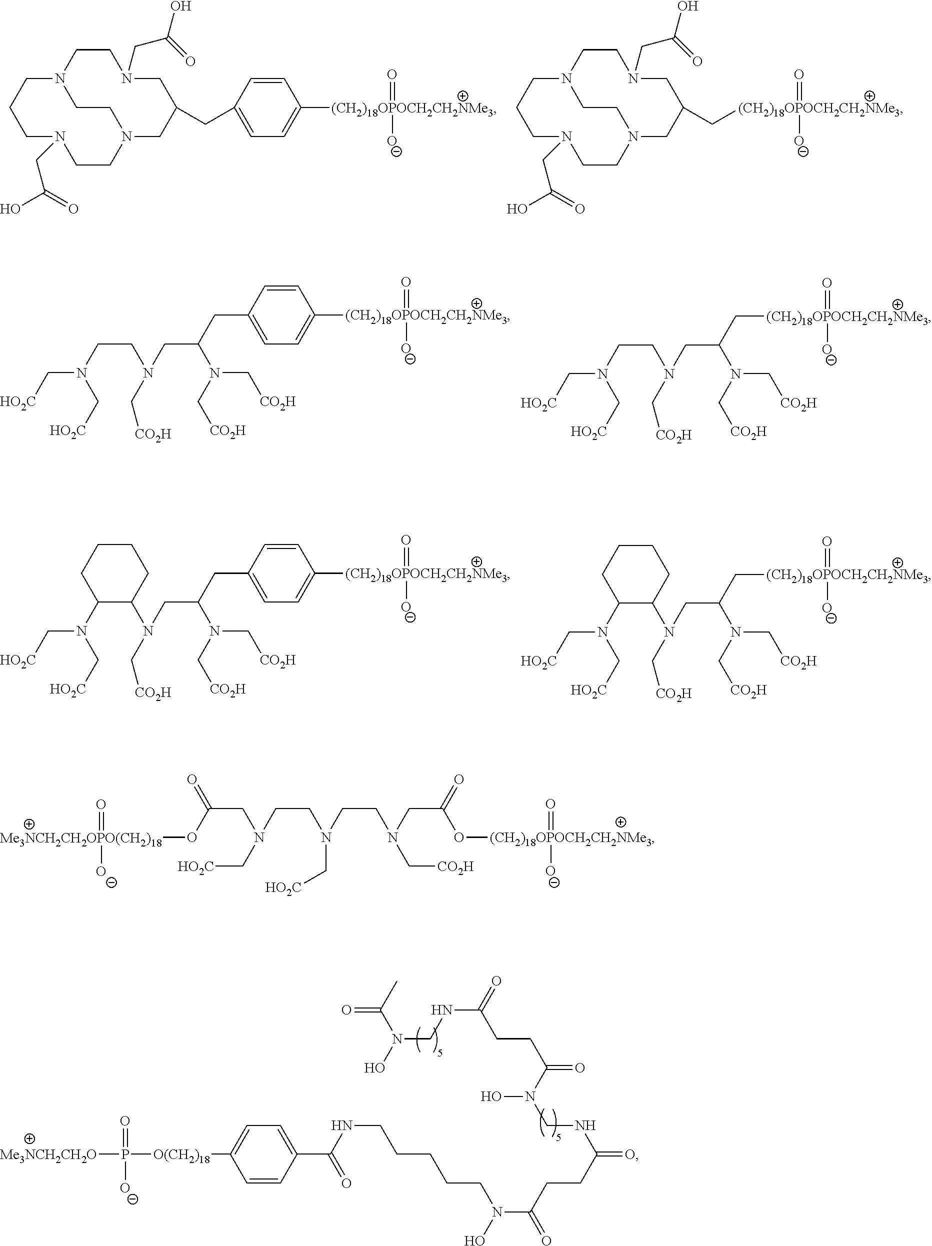

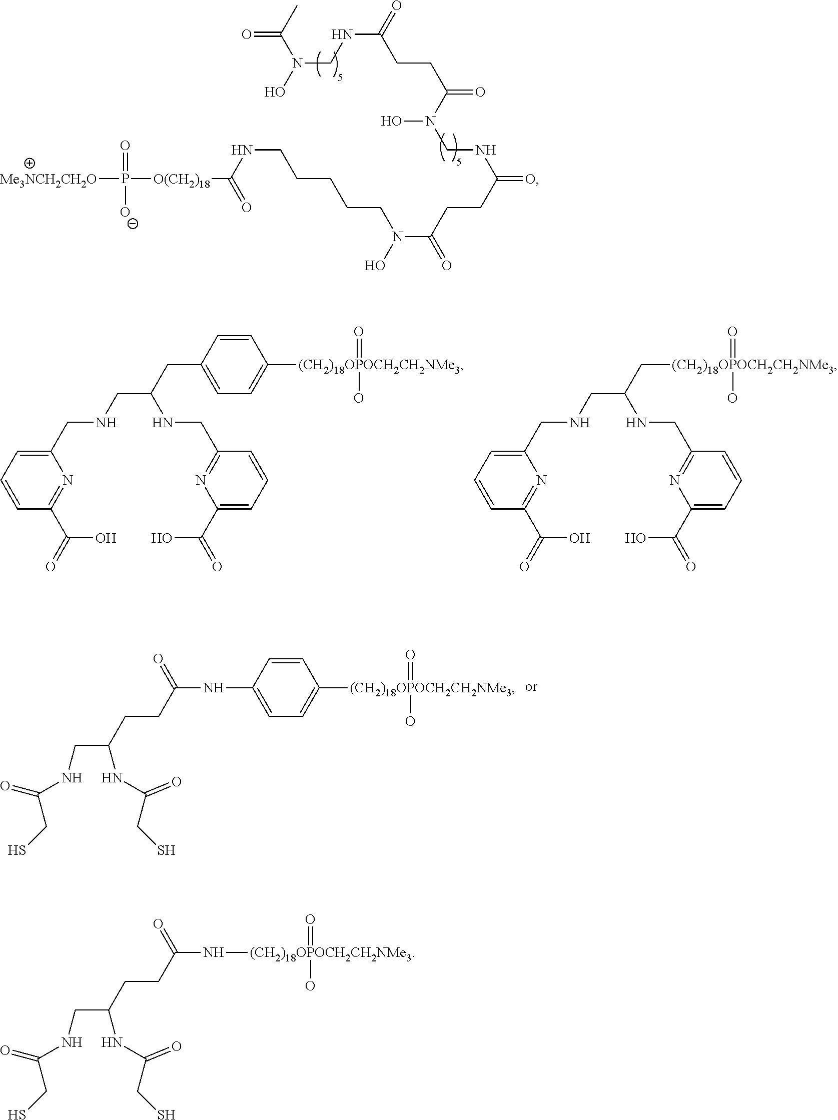

In some embodiments, the contrast agent has the chemical structure:

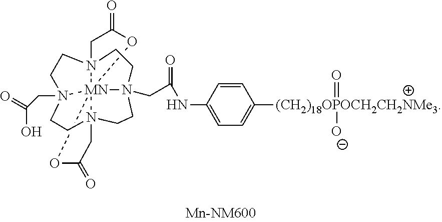

In some such embodiments, Mn is Mn-51 or Mn-52.

In a second aspect, this disclosure encompasses a composition that includes a dual-modality contrast agent, as described above, and a pharmaceutically acceptable carrier. The disclosed contrast agents are more hydrophilic (i.e., have higher solubility in water) than related halogenated compounds. Accordingly, in some embodiments, the composition does not include a surfactant.

In a third aspect, this disclosure encompasses a method for detecting or imaging one or more cancer tumor cells in a biological sample. The method includes the steps of (a)

contacting the biological sample with a dual-modality contrast agent, as described above; and (b) identifying individual cells or regions within the biological sample that are emitting signals characteristic of the chelated Mn²⁺ isotope, whereby one or more cancer tumor cells are detected or imaged.

In some embodiments, the step of identifying individual cells or regions within the biological sample that are emitting signals characteristic of the chelated Mn²⁺ isotope is performed using magnetic resonance imaging (MRI) or using both MRI and positron emission topography (PET) imaging.

In some embodiments, the biological sample is part or all of a subject. In some such embodiments, the contacting step is performed by injecting the contrast agent into the subject. In some such embodiments, the injection is performed intravenously.

In some embodiments, the subject is a human.

In some embodiments, the step of identifying individual cells or regions within the biological sample that are emitting signals characteristic of the chelated Mn²⁺ isotope is performed using both MRI and PET, and the dual-modality contrast agent as described above is the contrast agent that is used for both the MRI and PET. In some such embodiments, the MRI and PET imaging are performed simultaneously.

In some embodiments, the PET imaging is performed simultaneously with imaging the biological sample using computerized tomography (CT) imaging (PET/CT).

In some embodiments, the cancer cells are adult solid tumor cells or pediatric solid tumor cells.

In some embodiments, the cancer cells are melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, or pediatric brain tumor cells.

In a fourth aspect, this disclosure encompasses a method of diagnosing cancer in a subject. The method includes performing the method of detecting and/or imaging cancers cells, as described above, wherein the biological sample is obtained from, part of, or all of a subject. If cancer cells are detected or imaged as a result, the subject is diagnosed with cancer.

In some embodiments, the cancer that is diagnosed is an adult solid tumor or a pediatric solid tumor.

In some embodiments, the cancer is melanoma, neuroblastoma, lung cancer, adrenal cancer, colon cancer, colorectal cancer, ovarian cancer, prostate cancer, liver cancer, subcutaneous cancer, squamous cell cancer, intestinal cancer, retinoblastoma, cervical cancer, glioma, breast cancer, pancreatic cancer, Ewings sarcoma, rhabdomyosarcoma, osteosarcoma, retinoblastoma, Wilms' tumor, or a pediatric brain tumor.

In a fifth aspect, this disclosure encompasses a method of monitoring the efficacy of a cancer therapy in a human subject. The method includes the steps of performing the detection and/or imaging method as described above at two or more different times on the biological sample, wherein the biological sample is obtained from, part of, or all of a subject, and whereby the change in strength of the signals characteristic of the Mn²⁺ isotope between the two or more different times is correlated with the efficacy of the cancer therapy.

In some embodiments, the cancer therapy being monitored is chemotherapy or radiotherapy.

Other objects, features and advantages of the present invention will become apparent after review of the specification, claims and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the chemical structure of a previously-disclosed iodinated compound containing the APC backbone, 18-(p-iodophenyl) octadecyl phosphocholine (NM404).

FIG. 2 shows a time course MRI image of a tumor-bearing mouse following injection of Gd-NM600 showing enhancement of the tumor (T) by 24 hours.

FIG. 3 shows the chemical structure of an exemplary alkylphosphocholine Mn chelate that can be used as a dual modality PET/MRI contrast agent (Mn-NM600).

FIG. 4 includes PET/CT images for an HT-29 mouse from scans taken 4 hours (left panel) and 1 day (right panel) post-injection with ⁵²Mn-NM600. The images show tissue activity calculated as a percent of injected dose/g tissue (% ID/g, scale shown on far right).

FIG. 5 includes PET/CT images for a PC-3 mouse from scans taken 4 hours (left panel) and 1 day (right panel) post-injection with ⁵²Mn-NM600. The images show tissue activity calculated as a percent of injected dose/g tissue (% ID/g, scale shown to the right of each image).

FIG. 6 includes PET/CT images for an HT-29 mouse from scans taken 2 days (left panel), 3 days (second panel from the left), 5 days (second panel form the right) and 7 days (right panel) post-injection with ⁵²Mn-NM600. The images show tissue activity calculated as a percent of injected dose/g tissue (% ID/g, scale shown to the right of the images).

FIG. 7 includes PET/CT images for a PC-3 mouse from scans taken 2 days (left panel), 3 days (second panel from the left), 5 days (second panel form the right) and 7 days (right panel) post-injection with ⁵²Mn-NM600. The images show tissue activity calculated as a percent of injected dose/g tissue (% ID/g, scale shown to the right of the images).

FIG. 8 is a graph showing PET quantitative region of interest data (chelate uptake as a function of time) for HT-29 tumor tissue and healthy heart, liver and muscle tissue in HT-29 mice injected with ⁵²Mn-NM600.

FIG. 9 is a graph showing PET quantitative region of interest data (chelate uptake as a function of time) for PC3 tumor tissue and healthy heart, liver and muscle tissue in PC3 mice injected with ⁵²Mn-NM600.

FIG. 10 is a bar graph illustrating ex vivo chelate biodistribution in healthy and tumor tissues in both PC3 and HT-29 mice 48 hours post-injection of ⁵²Mn-NM600.

FIG. 11 includes simultaneous PET/CT (center panels) and MR images (left panels), as well as a combined PET/MR images (right panels) of a rat bearing U87MG tumors in the lower flank at days 1 (top row) and 5 (bottom row) after co-injection of Mn-NM600 and Gd-MN600. Excellent co-registration of the PET and MRI-enhanced tumor signal can be observed. The yellow arrow points to the tumor.

FIG. 12 is a bar graph illustrating ex vivo chelate biodistribution in healthy and tumor tissues in U87MG mice on day 5 post-injection of ⁵²Mn-NM600.

FIG. 13 includes MR images of a Balb/C mouse bearing 4T1 breast tumor tumors in the flank (arrows) before injection (top row) and 24 hours after injection (bottom row) with 3 mg of two different forms of the manganese-based chelates: Mn-NM600 (left column; where the chelating agent is DOTA) and Mn-NM620 (right column; where the chelating agent is NOTA). Tumor signal is enhanced 24 hours post-administration for both Mn-chelates.

### DETAILED DESCRIPTION

### I. IN GENERAL

This disclosure is not limited to the particular methodology, protocols, materials, and reagents described, as these may vary. The terminology used herein is for the purpose of describing particular embodiments only, and is not intended to limit the scope of the present invention, which will be limited only by any later-filed nonprovisional applications.

As used herein and in the appended claims, the singular forms “a”, “an”, and “the” include plural reference unless the context clearly dictates otherwise. The terms “a” (or “an”), “one or more” and “at least one” can be used interchangeably herein. The terms “comprising” and variations thereof do not have a limiting meaning where these terms appear in the description and claims. Accordingly, the terms “comprising”, “including”, and “having” can be used interchangeably.

Unless defined otherwise, all technical and scientific terms used herein have the same meanings as commonly understood by one of ordinary skill in the art to which the disclosed subject matter belongs. Although any methods and materials similar or equivalent to those described herein can be used in the practice or testing of the present invention, exemplary methods and materials are now described.

All publications and patents specifically mentioned herein are incorporated by reference for all purposes, including describing and disclosing the chemicals, instruments, statistical analysis and methodologies which are reported in the publications which might be used in connection with the disclosed subject matter. All references cited in this specification are to be taken as indicative of the level of skill in the art.

The terminology as set forth herein is for description of the exemplary embodiments only, and should not be construed as limiting of the invention as a whole. Unless otherwise specified, “a,” “an,” “the,” and “at least one” are used interchangeably and mean one or more than one.

The disclosure is inclusive of the compounds described herein (including intermediates) in any of their pharmaceutically acceptable forms, including isomers (e.g., diastereomers and enantiomers), tautomers, salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

“Pharmaceutically acceptable” as used herein means that the compound or composition or carrier is suitable for administration to a subject to achieve the results of the clinical testing (e.g., detection and/or imaging) described herein, without unduly deleterious side effects.

As used herein, “pharmaceutically-acceptable carrier” includes any and all dry powder, solvents, dispersion media, coatings, antibacterial and antifungal agents, isotonic agents, absorption delaying agents, and the like. Pharmaceutically-acceptable carriers are materials, useful for the purpose of administering the compounds in the method of the present invention, which are preferably non-toxic, and may be solid, liquid, or gaseous materials, which are otherwise inert and pharmaceutically acceptable, and are compatible with the compounds of the present invention. Examples of such carriers include, without limitation, various lactose, mannitol, oils such as corn oil, buffers such as PBS, saline, polyethylene glycol, glycerin, polypropylene glycol, dimethylsulfoxide, an amide such as dimethylacetamide, a protein such as albumin, and a detergent such as Tween 80, mono- and oligopolysaccharides such as glucose, lactose, cyclodextrins and starch.

The term “administering” or “administration,” as used herein, refers to providing the disclosed compounds or pharmaceutical compositions to a subject suffering from or at risk of the diseases or conditions to be detected and/or monitored.

A route of administration in pharmacology is the path by which a drug is taken into the body. Routes of administration may be generally classified by the location at which the substance is applied. Common examples may include oral and intravenous administration. Routes can also be classified based on where the target of action is. Action may be topical (local), enteral (system-wide effect, but delivered through the gastrointestinal tract), or parenteral (systemic action, but delivered by routes other than the GI tract), via lung by inhalation. One form of local administration referred to in this submission is intratumoral (IT), whereby an agent is injected directly into, or adjacent to, a known tumor site.

A topical administration emphasizes local effect, and substance is applied directly where its action is desired. Sometimes, however, the term topical may be defined as applied to a localized area of the body or to the surface of a body part, without necessarily involving target effect of the substance, making the classification rather a variant of the classification based on application location. In an enteral administration, the desired effect is systemic (non-local), substance is given via the digestive tract. In a parenteral administration, the desired effect is systemic, and substance is given by routes other than the digestive tract.

Non-limiting examples for topical administrations may include epicutaneous (application onto the skin), e.g., allergy testing or typical local anesthesia, inhalational, e.g. asthma medications, enema, e.g., contrast media for imaging of the bowel, eye drops (onto the conjunctiva), e.g., antibiotics for conjunctivitis, ear drops, such as antibiotics and corticosteroids for otitis externa, and those through mucous membranes in the body.

Enteral administration may be administration that involves any part of the gastrointestinal tract and has systemic effects. The examples may include those by mouth (orally), many drugs as tablets, capsules, or drops, those by gastric feeding tube, duodenal feeding tube, or gastrostomy, many drugs and enteral nutrition, and those rectally, various drugs in suppository.

Examples of parenteral administrations may include intravenous (into a vein), e.g. many drugs, total parenteral nutrition intra-arterial (into an artery), e.g., vasodilator drugs in the treatment of vasospasm and thrombolytic drugs for treatment of embolism, intraosseous infusion (into the bone marrow), intra-muscular, intracerebral (into the brain parenchyma), intracerebroventricular (into cerebral ventricular system), intrathecal (an injection into the spinal canal), and subcutaneous (under the skin). Among them, intraosseous infusion is, in effect, an indirect intravenous access because the bone marrow drains directly into the venous system. Intraosseous infusion may be occasionally used for drugs and fluids in emergency medicine and pediatrics when intravenous access is difficult.

The following abbreviations are used in this disclosure:

APC, alkylphosphocholine.

CEMRI, contrast-enhanced magnetic resonance imaging.

CT, computed tomography.

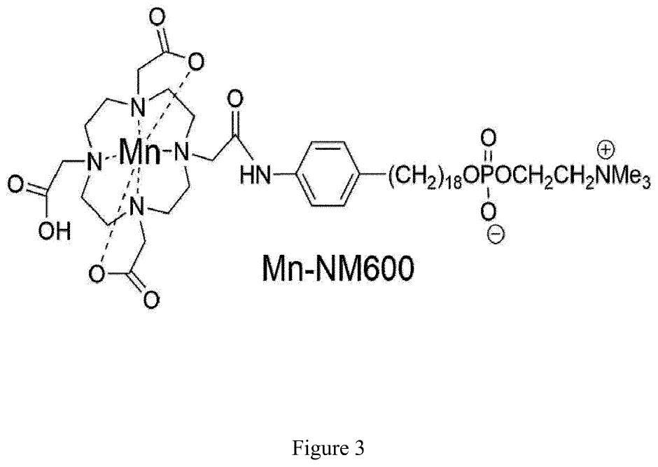

Gd-NM-600, a Gd-chelated phospholipid ether as shown in FIG. 3, except that the Mn atom is substituted with a Gd atom. Gd-NM-600 is selectively taken up and retained by cancerous tumors, and is used as a cancer-targeting MRI contrast agent in the studies disclosed in the examples.

Mn-NM600, the Mn-chelated phospholipid ether shown in FIG. 3, which is selectively taken up and retained by cancerous tumors and used as a PET contrast agent or a dual PET/MRI contrast agent in the studies disclosed in the examples.

MR, magnetic resonance.

MRI, magnetic resonance imaging.

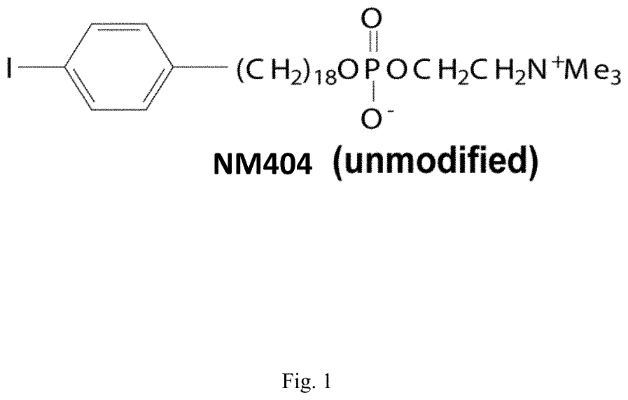

NM404, the iodinated phospholipid ether shown in FIG. 1. NM404 has previously been shown to be selectively taken up and retained by cancerous tumors.

PET, positron emission tomography.

PLE, phospholipid ether.

### II. THE INVENTION

This disclosure is directed to compounds having a cancer tumor-targeting alkylphosphocholine (APC) backbone chelated to a Mn²⁺ isotope, wherein the Mn²⁺ isotope is preferably chelated to the APC backbone through a macrocyclic chelating moiety. The compounds, which are differentially taken up and retained by a variety of cancerous solid tumor cells, can used as tumor-targeting dual modality contrast agents for both contrast enhanced magnetic resonance imaging (CEMRI) and positron emission tomography (PET). In some embodiments, CEMRI and PET imaging in a subject can be performed simultaneously after injection of a subject with the disclosed contrast agents.

The disclosed compounds target tumor cells with great specificity, and the chelated Mn²⁺ isotope is stably bound to the chelating moiety. Furthermore, they are cleared by the liver rather than the kidneys. Accordingly, the disclosed agents provide a safer alternative to Gd-based MRI contrast agents that differentially targets malignant tumors. The use of these contrast agents will both significantly improve MRI-based cancer detection and diagnosis, and will open up new avenues for the implementation of combined PET/MRI cancer detection and diagnosis.

A. Manganese Chelates of PLE Analogs for MRI and PET Detection/Imaging

The disclosed dual modality contrast agents utilize a cancer-targeting alkylphosphocholine (APC) carrier backbone, along with a chelating moiety to which a Mn²⁺ isotope is chelated. The chelating moiety binds very tightly to the chelated Mn²⁺, and the resulting contrast agent is extremely stable. In certain embodiments, the chelating moiety is a macrocyclic chelating moiety.

Mn²⁺ possesses the nuclear and magnetic properties necessary for use as an MRI contrast agent. Thus, the dual-modality contrast agents can be used as tumor-specific MRI contrast agent for general broad spectrum tumor imaging and characterization. In addition, the contrast agents are suitable for use in MRI-based therapy response monitoring to both chemotherapy and radiotherapy.

For use as a tumor-specific PET contrast agent, the chelated Mn²⁺ may be a positron-emitting Mn isotope, such as Mn-51 or Mn-52. Such dual-modality contrast agents can also be used as tumor-specific PET contrast agent for general broad spectrum tumor imaging and characterization, and as a single contrast agent for simultaneous MRI/PET imaging.

B. Methods of Synthesizing Exemplary M-PLE Analogs

The synthesis of several different exemplary compounds containing metal-chelating moieties is outlined below. Once such compounds are synthesized, they can be readily chelated with a variety of metal ions, including Mn²⁺.

The Proposed synthesis of compound 1 is shown below. The first step of the synthesis is similar to described in Org Synth, 2008, 85, 10-14. The synthesis is started from cyclen which is converted into DO3A tris-Bn ester. This intermediate is then conjugated with NM404 in the presence of the base and Pd catalyst. Finally, benzyl protecting groups are removed by the catalytic hydrogenation.

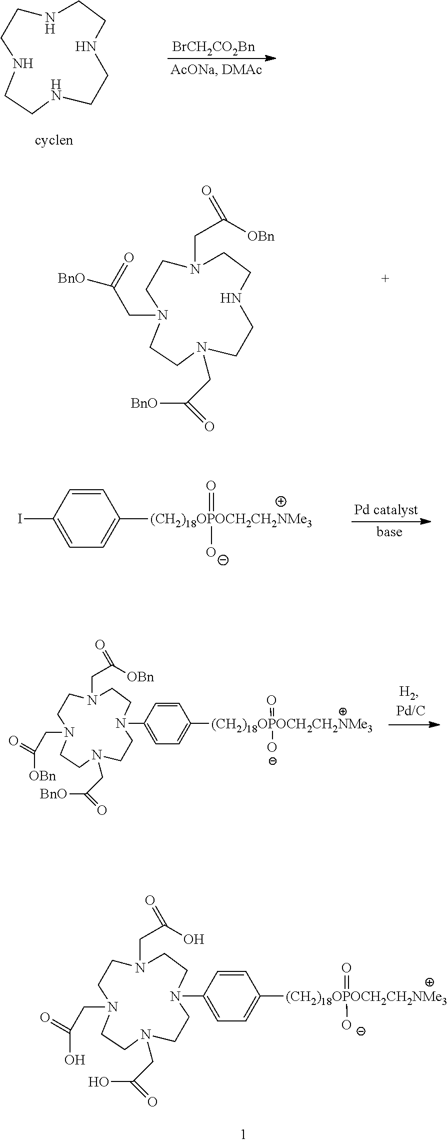

Synthesis of compound 2 is shown below. It begins with DO3A tris-Bn ester which is alkylated with 3-(bromo-prop-1-ynyl)-trimethylsilane. After alkylation, the trimethylsilyl group is removed and the intermediate acetylene is coupled with NM404 by the Sonogashira reaction. The benzyl groups are removed and the triple bond is hydrogenated simultaneously in the last step of the synthesis.

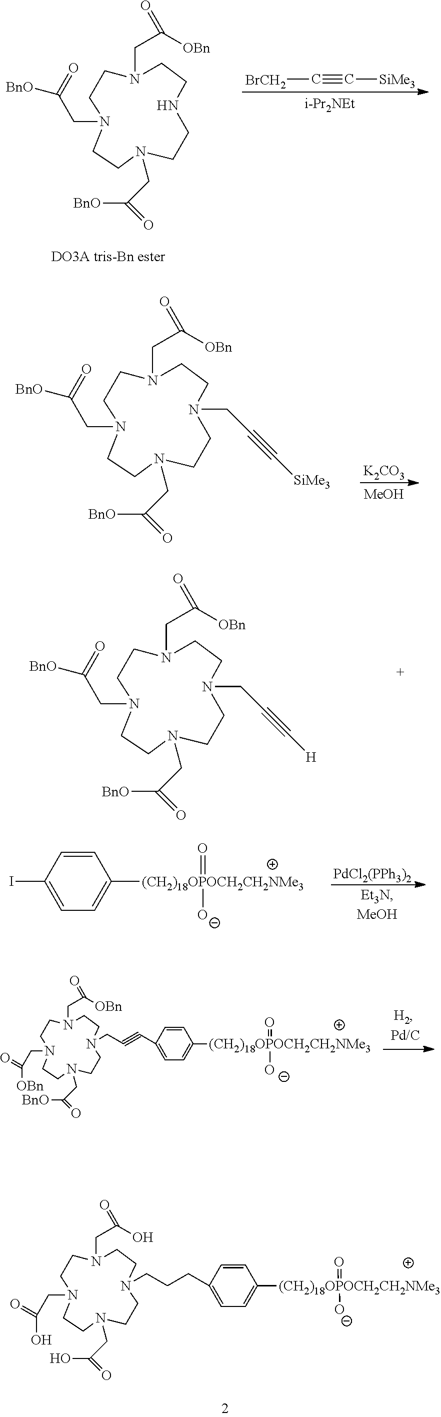

Compounds 5 and 6 can be synthesized from same precursors, DTPA dianhydride and 18-p-(3-hydroxyethyl-phenyl)-octadecyl phosphocholine as shown in the schemes below.

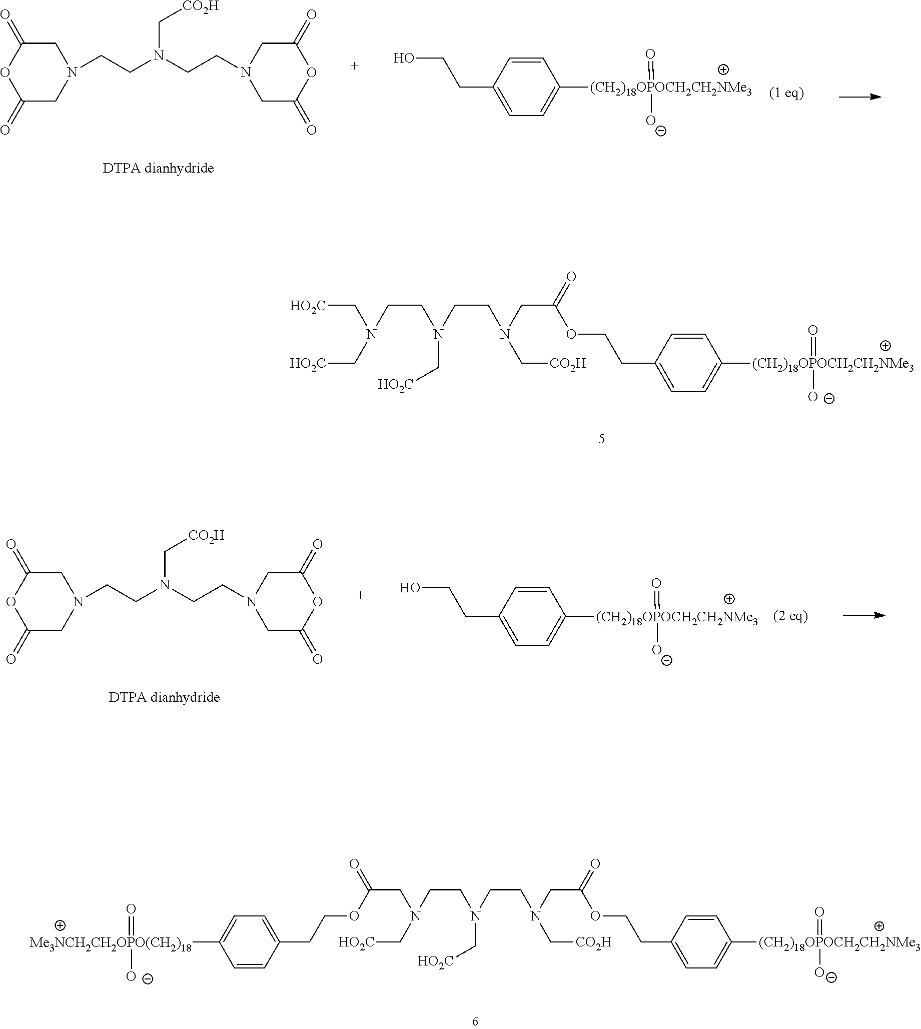

NOTA-NM404 conjugates can be synthesized in an analogous manner. One example of NOTA-NM404 conjugate 7:

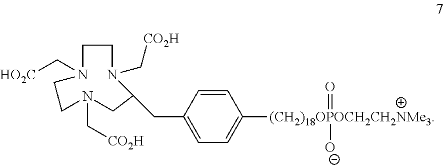

C. Dosage Forms and Administration Methods

MRI and PET contrast agents are most commonly injected intravenously. However, other administration routes (topical or systemic) can also be used.

In certain embodiments, the disclosed contrast agents may be provided as pharmaceutically acceptable salts. Other salts may, however, be useful in the preparation of the alkylphosphocholine analogs or of their pharmaceutically acceptable salts. Suitable pharmaceutically acceptable salts include, without limitation, acid addition salts which may, for example, be formed by mixing a solution of the alkylphosphocholine analog with a solution of a pharmaceutically acceptable acid such as hydrochloric acid, sulfuric acid, methanesulfonic acid, fumaric acid, maleic acid, succinic acid, acetic acid, benzoic acid, oxalic acid, citric acid, tartaric acid, carbonic acid or phosphoric acid.

Where the disclosed contrast agents have at least one asymmetric center, they may accordingly exist as enantiomers. Where the disclosed contrast agents possess two or more asymmetric centers, they may additionally exist as diastereoisomers. All such isomers and mixtures thereof in any proportion are encompassed within the scope of the present disclosure.

The disclosure also includes methods of using pharmaceutical compositions comprising one or more of the disclosed contrast agents in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for parenteral, intranasal, sublingual or rectal administration, or for administration by inhalation or insufflation.

The liquid forms in which the contrast agents may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium caboxymethylcellulose, methylcellulose, polyvinylpyrrolidone or gelatin.

The contrast agents are more hydrophilic than the corresponding iodinated analogs. Thus, the liquid form may lack a surfactant, or have a much smaller amount of surfactant than is used with injectable forms of other APC analogs.

The disclosed contrast agents are particularly useful when formulated in the form of a pharmaceutical injectable dosage, including in combination with an injectable carrier system. As used herein, injectable and infusion dosage forms (i.e., parenteral dosage forms) include, but are not limited to, liposomal injectables or a lipid bilayer vesicle having phospholipids that encapsulate an active substance. Injection includes a sterile preparation intended for parenteral use.

Five distinct classes of injections exist as defined by the USP: emulsions, lipids, powders, solutions and suspensions. Emulsion injection includes an emulsion comprising a sterile, pyrogen-free preparation intended to be administered parenterally. Lipid complex and powder for solution injection are sterile preparations intended for reconstitution to form a solution for parenteral use. Powder for suspension injection is a sterile preparation intended for reconstitution to form a suspension for parenteral use. Powder lyophilized for liposomal suspension injection is a sterile freeze dried preparation intended for reconstitution for parenteral use that is formulated in a manner allowing incorporation of liposomes, such as a lipid bilayer vesicle having phospholipids used to encapsulate an active drug substance within a lipid bilayer or in an aqueous space, whereby the formulation may be formed upon reconstitution. Powder lyophilized for solution injection is a dosage form intended for the solution prepared by lyophilization (“freeze drying”), whereby the process involves removing water from products in a frozen state at extremely low pressures, and whereby subsequent addition of liquid creates a solution that conforms in all respects to the requirements for injections. Powder lyophilized for suspension injection is a liquid preparation intended for parenteral use that contains solids suspended in a suitable fluid medium, and it conforms in all respects to the requirements for Sterile Suspensions, whereby the medicinal agents intended for the suspension are prepared by lyophilization. Solution injection involves a liquid preparation containing one or more drug substances dissolved in a suitable solvent or mixture of mutually miscible solvents that is suitable for injection.

Solution concentrate injection involves a sterile preparation for parenteral use that, upon addition of suitable solvents, yields a solution conforming in all respects to the requirements for injections. Suspension injection involves a liquid preparation (suitable for injection) containing solid particles dispersed throughout a liquid phase, whereby the particles are insoluble, and whereby an oil phase is dispersed throughout an aqueous phase or vice-versa. Suspension liposomal injection is a liquid preparation (suitable for injection) having an oil phase dispersed throughout an aqueous phase in such a manner that liposomes (a lipid bilayer vesicle usually containing phospholipids used to encapsulate an active drug substance either within a lipid bilayer or in an aqueous space) are formed. Suspension sonicated injection is a liquid preparation (suitable for injection) containing solid particles dispersed throughout a liquid phase, whereby the particles are insoluble. In addition, the product may be sonicated as a gas is bubbled through the suspension resulting in the formation of microspheres by the solid particles.

The parenteral carrier system includes one or more pharmaceutically suitable excipients, such as solvents and co-solvents, solubilizing agents, wetting agents, suspending agents, thickening agents, emulsifying agents, chelating agents, buffers, pH adjusters, antioxidants, reducing agents, antimicrobial preservatives, bulking agents, protectants, tonicity adjusters, and special additives.

D. Comparisons to Previously Disclosed Iodinated Compounds

In addition to containing a Mn²⁺ ion that can be used to increase the contrast of MRI images, there are other advantages to using Mn-chelated APCs to target cancer tumor tissue, rather than the previously disclosed iodinated analogs (see, e.g., FIG. 1).

Unlike iodinated analogs, APC chelates are too large to fit into known albumin binding pockets in the plasma and therefore exhibit different in vivo pharmacokinetic and biodistribution profiles. Lower binding energies lead to larger fractions of free molecule in the plasma, which affords more rapid tumor uptake. APC chelates also accumulate in tumors and clear from the blood much quicker than iodinated analog. Faster blood clearance is directly associated with lower bone marrow and off-target toxicity of radiopharmaceuticals. Faster clearance from normal tissues also improves imaging contrast.

APC chelates possess different physico-chemical characteristics than iodinated analogs. They are much more water-soluble, and therefore do not need surfactants to render them suitable for intravenous injection. APC chelates are based on ionic binding of the metal to the chelate, whereas iodinated compounds form covalent bonds with their carrier molecules. In vivo de-iodination is quite common in alkyl iodides whereas chelates tend to be extremely stable in vivo. Once de-iodination occurs, free iodide rapidly accumulates in the thyroid with a very long subsequent excretion half-life, whereas free metals are in general excreted from the body or detoxified much more quickly.

Finally, radiolabeled APC-metal chelates are easily labeled in nearly quantitative (>98%) yields under facile conditions, whereas radioiodination yields of iodinated analogs are much lower (typically about 50% for I-131 and 60% for I-124). Moreover, high specific activities can be achieved with chelates. Synthesis can be done using a radiolabeling kit in any nuclear pharmacy without the requirement of sophisticated ventilation equipment or training. In contrast, radioiodination must be done in a fume hood fitted with effluent monitoring equipment due to the volatility of radioactive iodine during the labeling reaction.

The following examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### III. EXAMPLES

### Introduction to the Examples

These examples demonstrate that (1) the disclosed contrast agents are differentially taken up by and retained by cancer tumor cells, (2) the disclosed contrast agents can be used as PET contrast agents to detect/image cancer in a subject, and (3) the disclosed contrast agents can be used as MRI contrast agents to detect/image cancer in a subject.

In Example 1, we provide an exemplary synthesis that could also be used to synthesize compounds chelating Mn²⁺ isotopes.

In Example 2, we demonstrate that an analog having a chelating moiety and chelated metal substituted for the iodine moiety of NM404 (Gd-NM600) is taken up by and can be used to facilitate the magnetic resonance imaging of solid tumor tissue, thus providing proof of concept for using the disclosed Mn²⁺ chelates as cancer-targeting MRI contrast agents.

In Example 3, we demonstrate that a similar analog having a chelating moiety chelated to a Mn²⁺ isotope (⁵²Mn-NM600) is taken up by and can be used to facilitate the positron emission tomography imaging of solid tumors in several in vivo models, thus providing additional proof of concept for using the disclosed metal chelates as cancer-targeting PET contrast agents.

In Example 4, we demonstrate the simultaneous MRI and PET imaging of a solid tumor in another in vivo model, where Gd-NM600 and ⁵²Mn-NM600 are injected simultaneously and used as the MRI and PET contrast agent, respectively. This example provides proof of concept for using the same chelate structure as a cancer-targeting dual-modality MRI/PET contrast agent.

In Example 5, we demonstrate the MRI imaging of a solid tumor in an in vivo model, using Mn-NM600 and Mn-NM620 as a MRI contrast agent. This example provides further proof of concept for using the Mn-chelates as a cancer-targeting dual-modality MRI/PET contrast agents.

### Example 1

### Synthesis of Metal Chelated NM600

In this Example, we show the synthetic scheme used to synthesize one exemplary phospholipid chelate, Gd-NM600. Analogs incorporating Mn isotopes could be synthesized in a similar manner, except that Mn is substituted for Gd.

Scheme for synthesizing Gd-NM600 (the disclosed radioactive metal isotopes could be substituted for Gd):

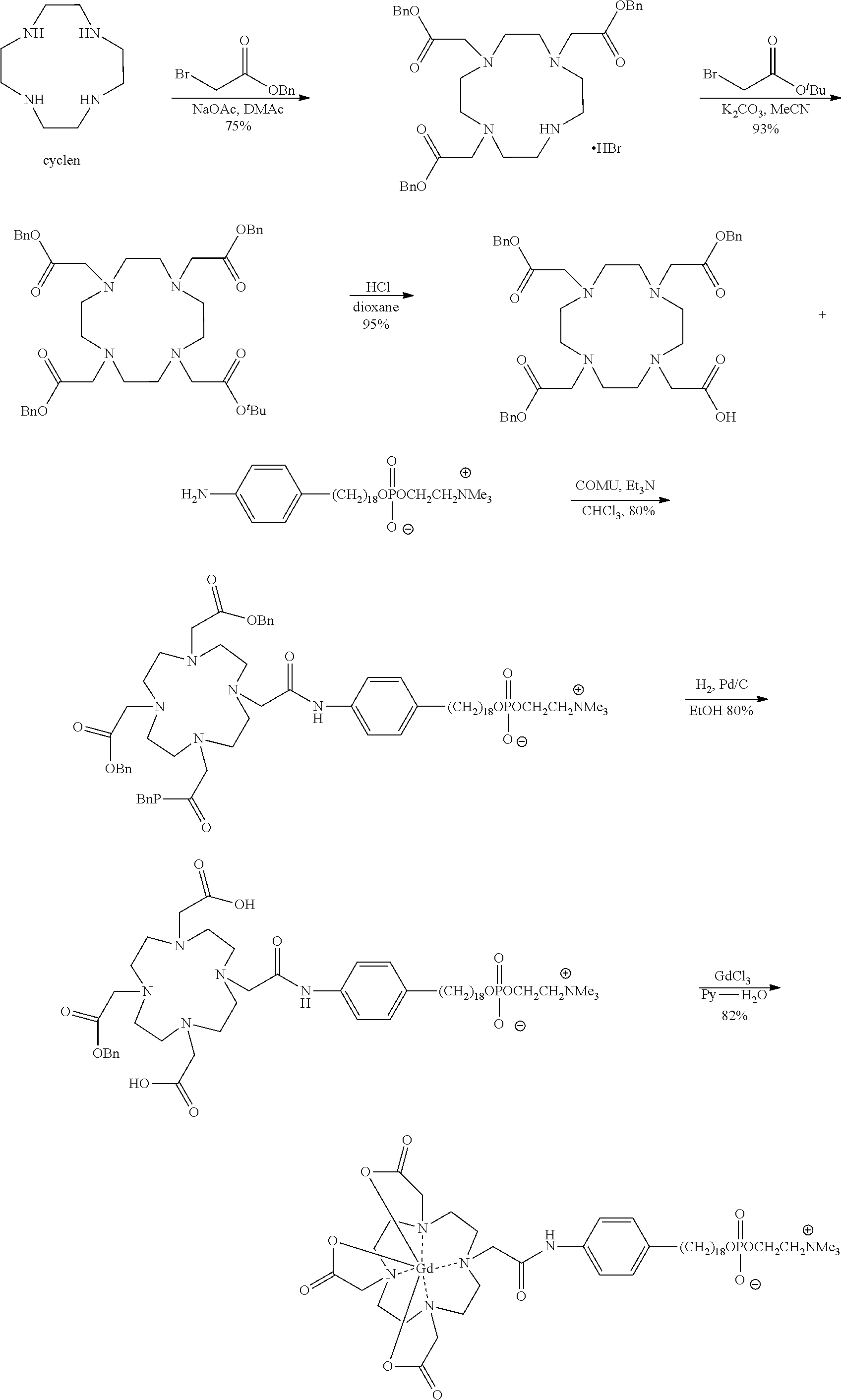

### Example 2

### In Vivo MRI Imaging Proof of Concept

In this example, we demonstrate the successful in vivo MRI imaging of a tumor, using Gd-NM600 as the MRI contrast agent. The data demonstrates that the backbone phospholipid and chelating moiety are taken up and retained by solid tumors, demonstrating that such chelates incorporating Mn²⁺ isotopes, as disclosed herein, would exhibit similar properties.

For proof-of-concept in vivo imaging of tumor uptake of the Gd-NM404 agent, nude athymic mouse with a flank A549 tumor (non small cell lung cancer) xenograft was scanned. The Gd-NM600 agent (2.7 mg) was delivered via tail vein injection. Mice were anesthetized and scanning performed prior to contrast administration and at 1, 4, 24, 48, and 72 hours following contrast delivery. Imaging was performed on a 4.7T Varian preclinical MRI scanner with a volume quadrature coil. T1-weighted images were acquired at all imaging time points using a fast spin echo scan with the following pulse sequence parameters: repetition time (TR)=206 ms, echo spacing=9 ms, echo train length=2, effective echo time (TE)=9 ms, 10 averages, with a 40×40 mm² field of view, 192×192 matrix, 10 slices of thickness 1 mm each.

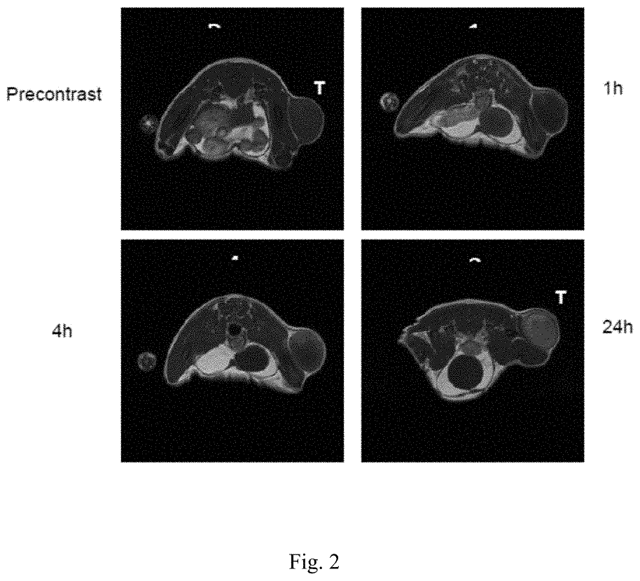

As seen in FIG. 2, MRI imaging of the tumor was significantly enhanced by 24 hours post-injection.

These results demonstrate that the differential uptake and retention of alkylphosphocholine analogs is maintained for the metal chelated analogs disclosed herein. Furthermore, the results demonstrate that such chelates containing metals known to have the properties necessary to facilitate increased MRI contrast (e.g., Gd or Mn) can be readily used as cancer tumor-targeting MRI contrast agents.

### Example 3

### In Vivo Uptake of ⁵²Mn-NM600 Metal Chelate in Mice Xenografted with Two Different Solid Tumor Types, Demonstrated by PET Imaging

In this example, we demonstrate the differential uptake of NM600 chelated with ⁵²Mn (Mn-NM600, see FIG. 3) in two different solid tumors in vivo, as demonstrated by PET/CT imaging of such tumors. These data provide additional support for the use of Mn-chelated alkylphosphocholine analogs as PET contrast agents.

Mice were each xenografted with two different solid tumor cell lines (PC-3 (prostate carcinoma) and HT-29 (colorectal adenocarcinoma). For each of the xenografted mice, cell suspension containing tumor cells was inoculated into subcutaneous tissue of one or both flanks of the mouse. Once xenograft tumors reached a minimum size, each mouse was injected with 150-300 μCi of NM600 radiolabeled with ⁵²Mn (⁵²Mn-NM600) via lateral tail vein injection. After an uptake period, PET imaging was performed in an Inveon micro PET/CT. Right before each scan, mice were anesthetized with isoflurane (2%) and placed in a prone position in the scanner. Longitudinal 40-80 million coincidence event static PET scans were acquired at 3, 12, 24, and 48 hours post-injection of the radiotracer and the images were reconstructed using an OSEM3D/MAP reconstruction algorithm.

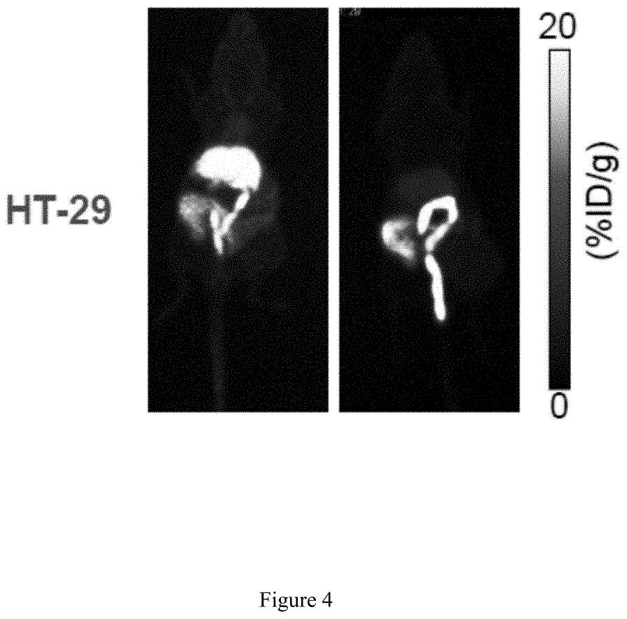

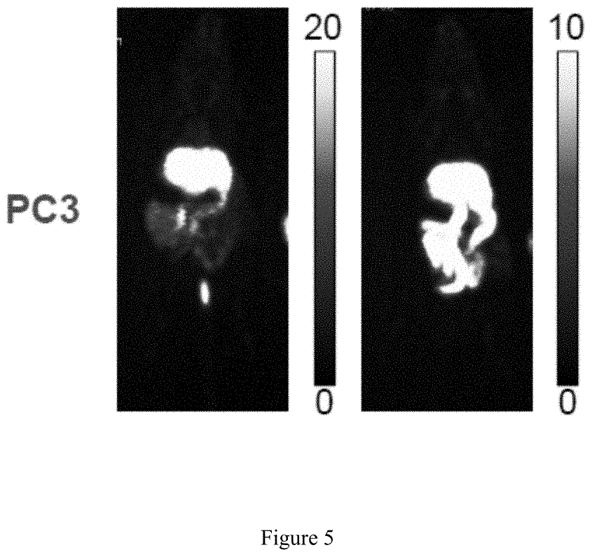

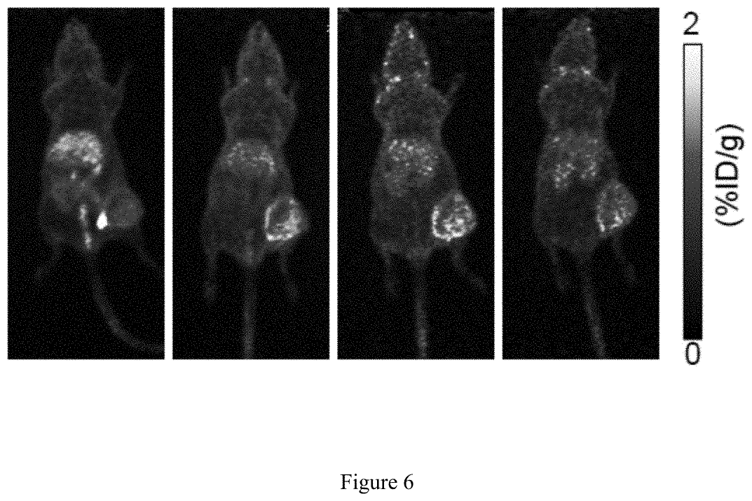

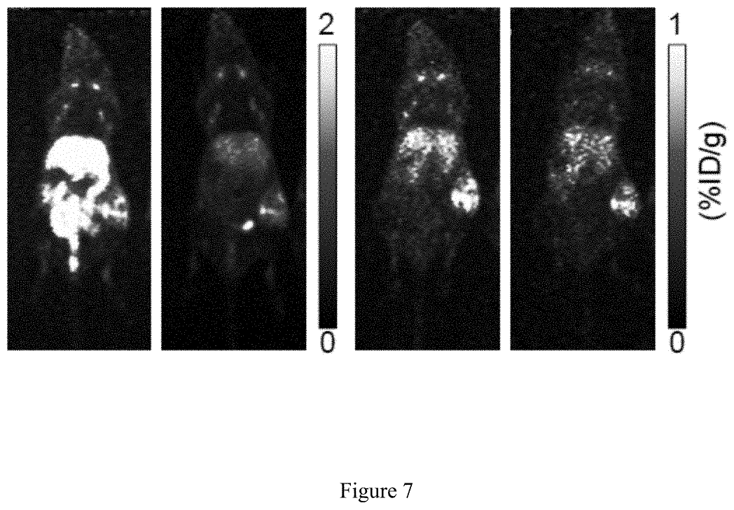

For HT-29 and PC3 mice injected with ⁵²Mn-NM600, PET images were obtained at 4 hours and one day post-injection (FIG. 4 for HT-29; FIG. 5 for PC3), as well as on days 2, 3, 5 and 7 post-injection (FIG. 6 for HT-29; FIG. 7 for PC-3).

As seen in FIGS. 4-7, the scanned mice produced PET/CT three-dimensional volume renderings showing cumulative absorbed dose distribution concentrated in the xenografted tumor. The results confirm the differential uptake of Mn-chelated NM600 into the xenografted solid tumor tissue, and demonstrate the potential use of Mn-NM600 and related analogs as cancer-targeting PET contrast agents.

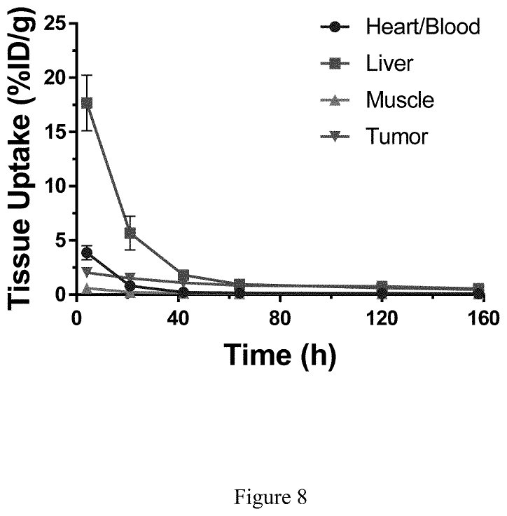

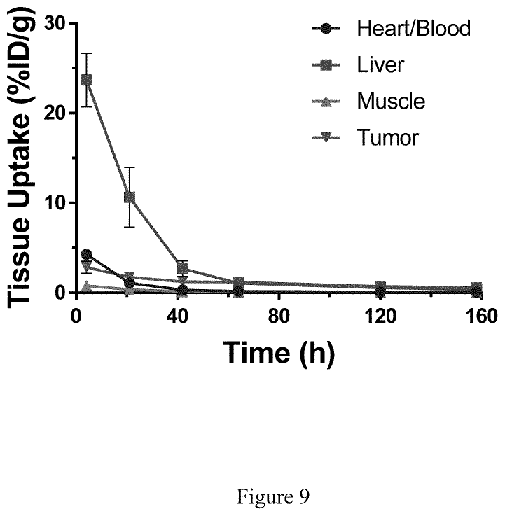

Quantitative region-of-interest analysis of the images was performed by manually contouring the tumor and other organs of interest. Quantitative data was expressed as percent injected dose per gram of tissue (% ID/g). Exemplary data show that both the HT-29 (FIG. 8) and PC3 tumor tissue (FIG. 9) effectively retained the ⁵²Mn-NM600 chelate, while healthy heart, liver, and muscle tissue all exhibited significantly decreased uptake/retention over time.

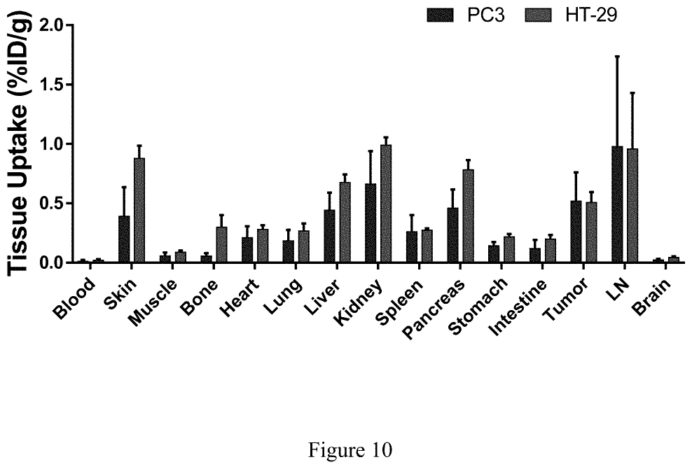

Ex vivo biodistribution analysis was performed after the last longitudinal PET scan. Mice were euthanized and tissues harvested, wet-weighed, and counted in an automatic gamma counter (Wizard 2480, Perkin Elmer). Exemplary biodistribution data show significant uptake and retention of ⁵²Mn-NM-600 in both tumor tissues (PC3 and HT-29, see FIG. 10).

Together, these results demonstrate that the disclosed Mn chelates can readily be used a cancer-targeting PET contrast agent.

### Example 4

### Simultaneous MRI and PET Imaging Using NM-600 Contrast Agents

In this example, we demonstrate the use of co-injected Gd-NM600 and ⁵²Mn-NM600 as a dual modality contrast agent in concurrently performed MRI and PET imaging in an in vivo solid tumor model (U87MG glioblastoma). The Gd-NM600 acts as the MRI contrast agent, and the ⁵²Mn-NM600 acts as the PET contrast agent. Given that Mn²⁺ is recognized as a viable alternative to Gd in MRI contrast agents, this example provides proof of principle for the use of ⁵²Mn-NM600 and related chelates as dual modality PET/MRI contrast agents.

Mice were xenografted with U87 MG glioblastoma solid tumor cell lines. For each of the xenografted mice, cell suspension containing tumor cells was inoculated into subcutaneous tissue of both flanks of the mouse. Once xenograft tumors reached a minimum size, each mouse was co-injected with ⁵²Mn-NM600 and Gd-NM600 via lateral tail vein injection. Simultaneous PET/CT and MRI scans were obtained at day 1 and day 5 post-injection.

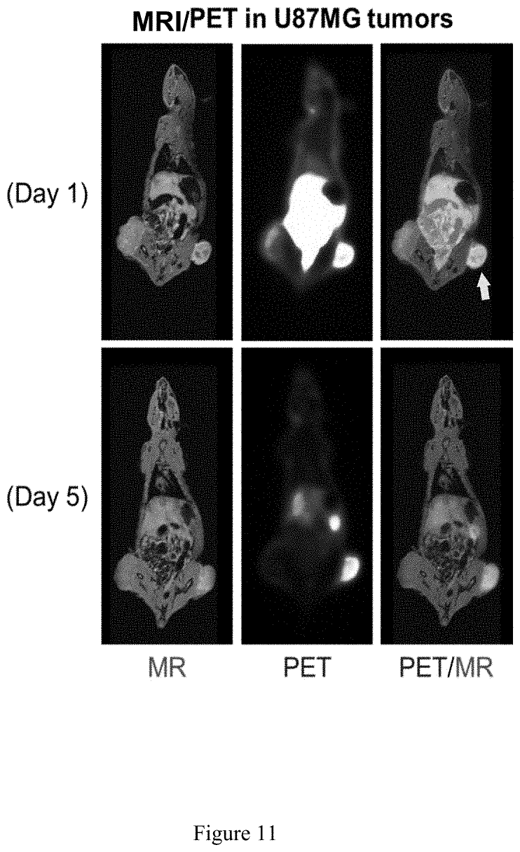

As seen in FIG. 11, excellent co-registration of the PET and MRI-enhanced tumor signal was observed. These results illustrate the potential of using NM-600 and related compounds, and Mn-chelated compounds specifically, as dual-modality cancer tumor-targeting PET and MRI contrast agents.

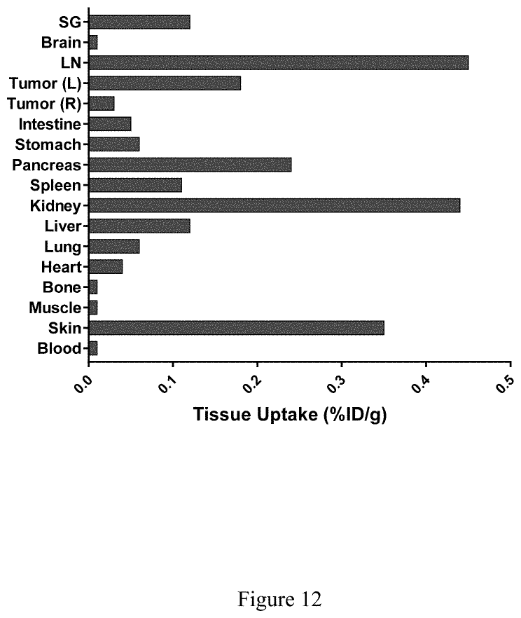

Ex vivo biodistribution analysis was performed after the last longitudinal PET scan (day 5 post-injection). Mice were euthanized and tissues harvested, wet-weighed, and counted in an automatic gamma counter (Wizard 2480, Perkin Elmer). Exemplary biodistribution data confirms significant uptake and retention of ⁵²Mn-NM-600 in tumor tissues (FIG. 12).

Together, these results demonstrate that the disclosed Mn chelates can readily be used as cancer-targeting dual modality MRI/PET contrast agents.

### Example 5

### The Mn-Chelates are Effective Tumor-Targeting MRI Contrast Agents

In this example, we demonstrate the use of Mn-NM600 (using DOTA as the chelating agent) and Mn-NM620 (using NOTA as the chelating agent) as a contrast agent in MRI in an in vivo mouse solid tumor model (4T1 breast tumor). Both of the chelates were shown to act as effective tumor-targeting MRI contrast agents. Accordingly, this example provides further proof of principle for the use of ⁵²Mn-NM600 and related chelates as dual modality PET/MRI contrast agents.

Balb/C mice were xenografted with 4T1 solid tumor cell lines. For each of the xenografted mice, cell suspension containing tumor cells was inoculated into subcutaneous tissue in the flank of the mouse. Once xenograft tumors reached a minimum size, each mouse was injected with 3 mg Mn-NM600 or Mn-NM620 via lateral tail vein injection. MRI scans (T1-weighted; 4.7T) were obtained before injection and at 24 hours post-injection.

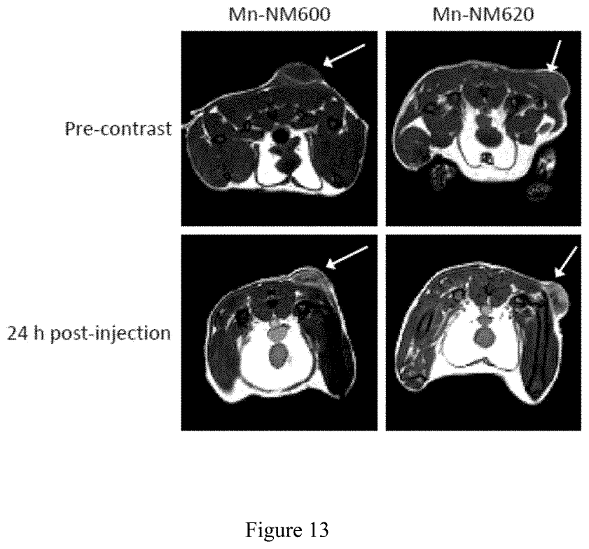

As seen in FIG. 13, an MRI-enhanced tumor signal was observed when using both Mn-chelates as MRI contrast agents. Together with the previously reported results using Mn-chelated compounds as a contrast agent in PET imaging, these results illustrate the potential of using NM-600 and related compounds, and Mn-chelated compounds specifically, as dual-modality cancer tumor-targeting PET and MRI contrast agents.

### Conclusion to the Examples

Currently, there are no effective contrast agents for the two high-resolution imaging modalities of PET and MRI that effectively target tumor tissue. These examples illustrate a new cancer-detecting/imaging strategy, using a single Mn-chelating and cancer-targeting alkylphosphocholine as a dual modality cancer-targeting MRI/PET contrast agent. This strategy addressed the toxicity concerns associated with the use of Gd-containing MRI contrast agents, while providing the additional advantage of strongly targeting cancer tissues.

Other embodiments and uses of the invention will be apparent to those skilled in the art from consideration from the specification and practice of the invention disclosed herein. All references cited herein for any reason, including all journal citations and U.S./foreign patents and patent applications, are specifically and entirely incorporated herein by reference. It is understood that the invention is not confined to the specific reagents, formulations, reaction conditions, etc., herein illustrated and described, but embraces such modified forms thereof as come within the scope of the following claims.

1. A dual-modality contrast agent having the formula: 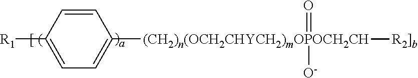
or a salt thereof, wherein:
R₁ comprises a chelating moiety that is chelated to a Mn²⁺ isotope;
a is 0 or 1;
n is an integer from 12 to 30;
m is 0 or 1;
Y is selected from the group consisting of —H, —OH, —COOH, —COOX, —OCOX, and —OX, wherein X is an alkyl or an arylalkyl;
R₂ is selected from the group consisting of —N⁺H₃, —N⁺H₂Z, —N⁺HZ₂, and —N⁺Z₃, wherein each Z is independently an alkyl or an aryl; and
b is 1 or 2.

2. The contrast agent of claim 1, wherein the chelating moiety is selected from the group consisting of 1,4,7,10-tetraazacyclododecane-1,4,7-triacetic acid (DO3A) and its derivatives; 1,4,7-triazacyclononane-1,4-diacetic acid (NODA) and its derivatives; 1,4,7-triazacyclononane-1,4,7-triacetic acid (NOTA) and its derivatives; 1,4,7,10-tetraazacyclododecane-1,4,7,10-tetraacetic acid (DOTA) and its derivatives; 1,4,7-triazacyclononane,1-glutaric acid-4,7-diacetic acid (NODAGA) and its derivatives; 1,4,7,10-tetraazacyclodecane,1-glutaric acid-4,7,10-triacetic acid (DOTAGA) and its derivatives; 1,4,8,11-tetraazacyclotetradecane-1,4,8,11-tetraacetic acid (TETA) and its derivatives; 1,4,8,11-tetraazabicyclo[6.6.2]hexadecane-4,11-diacetic acid (CB-TE2A) and its derivatives; diethylene triamine pentaacetic acid (DTPA), its diester, and its derivatives; 2-cyclohexyl diethylene triamine pentaacetic acid (CHX-A″-DTPA) and its derivatives; deforoxamine (DFO) and its derivatives; 1,2-[[6-carboxypyridin-2-yl]methylamino]ethane (H₂dedpa) and its derivatives; and DADA and its derivatives, wherein DADA comprises the structure:

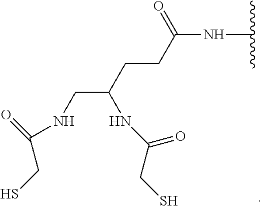 [SMILES: *NC(=O)CCC(CNC(=O)CS)NC(=O)CS]

3. The contrast agent of claim 1, wherein the chelating moiety that is chelated to the Mn²⁺ isotope is selected from the group consisting of:

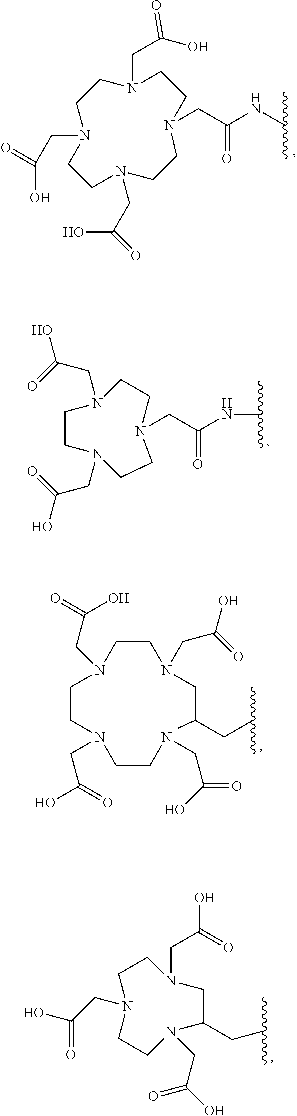 [SMILES: *NC(=O)CN1CCN(CC(=O)O)CCN(CC(=O)O)CCN(CC(=O)O)CC1 ; *NC(=O)CN1CCN(CC(=O)O)CCN(CC(=O)O)CC1 ; *CC(C)CN(CCN(CCN(CCNCC(=O)O)CC(=O)O)CC(=O)O)CC(=O)O ; *CC1CN(CC(=O)O)CCN(CC(=O)O)CCN1CC(=O)O]

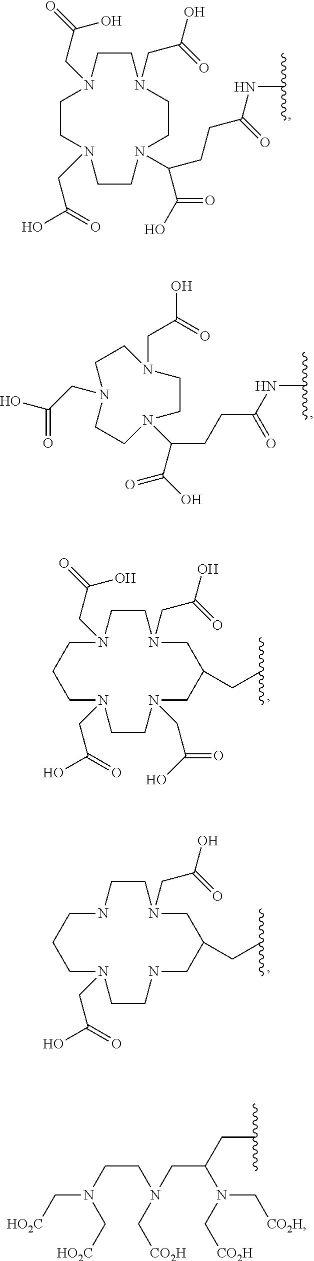

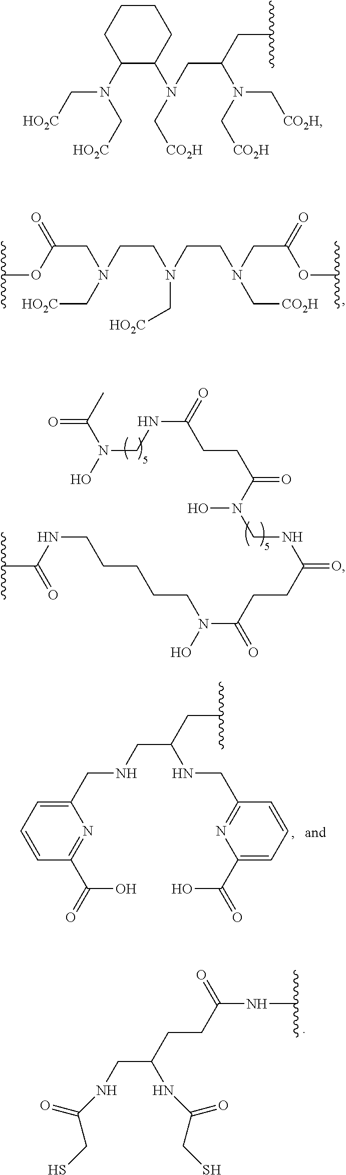

4. The contrast agent of claim 1, wherein the chelating moiety chelated to the Mn²⁺ isotope is selected from the group consisting of:

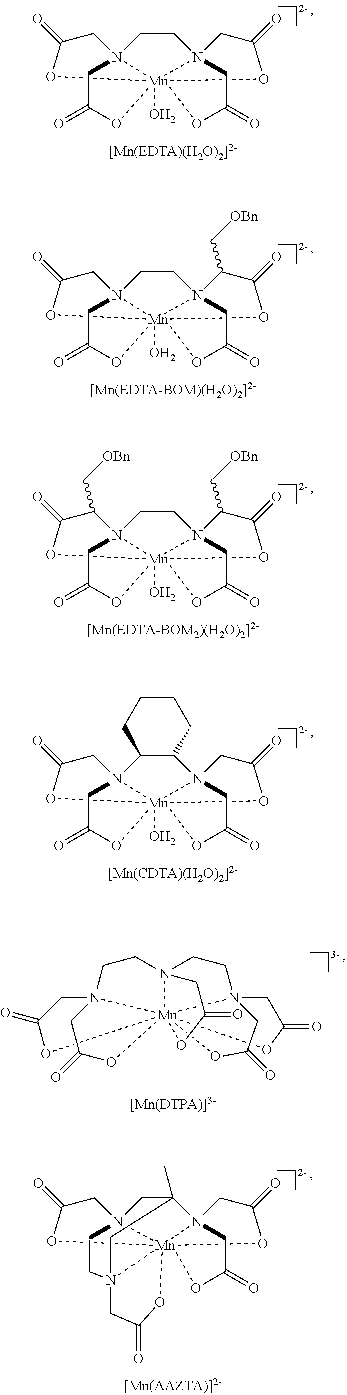

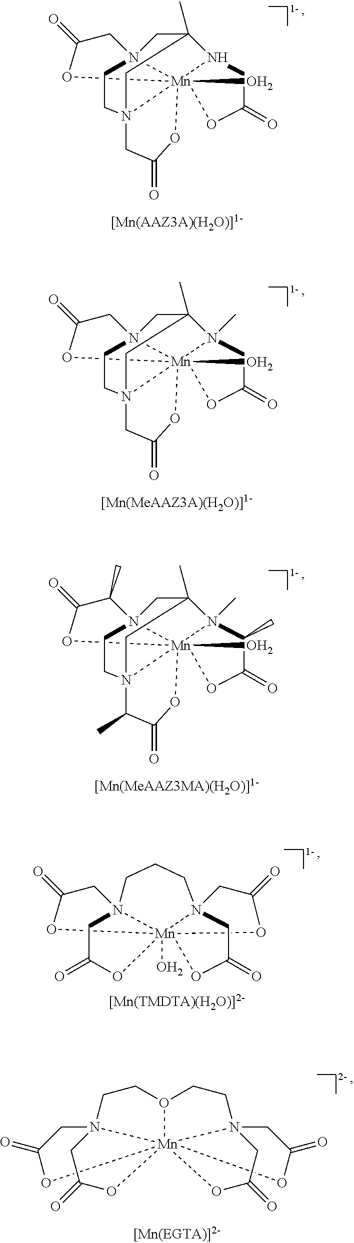

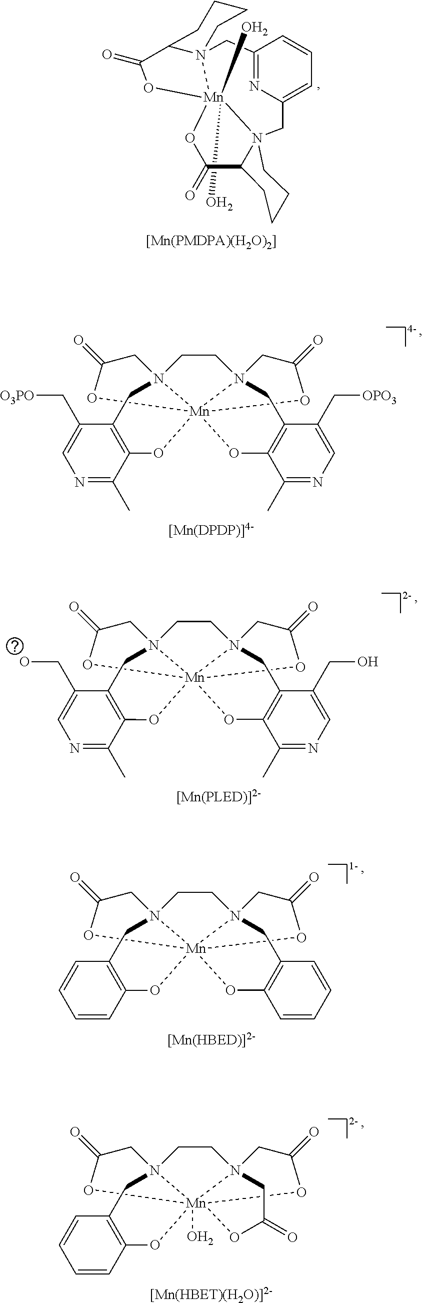

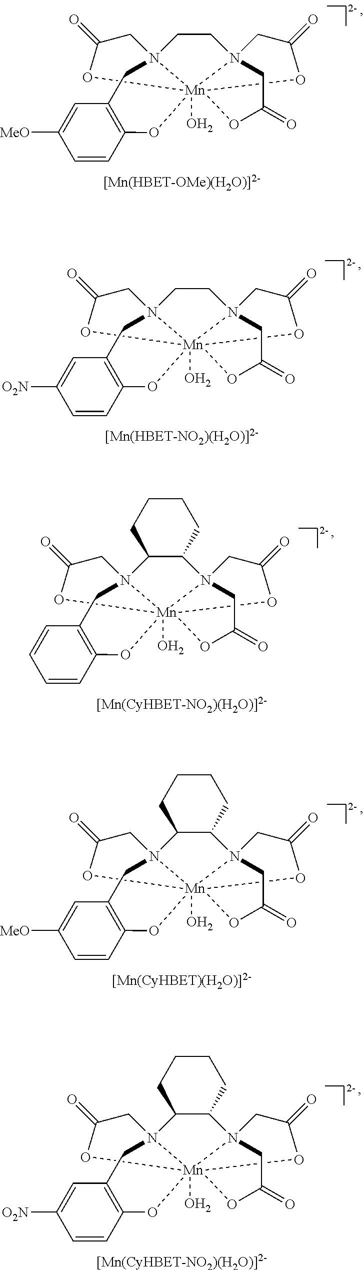

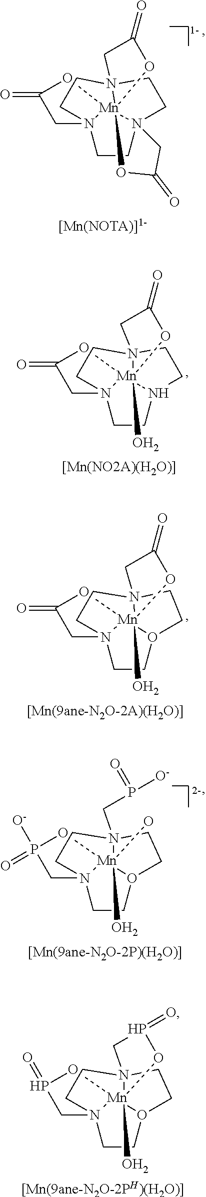

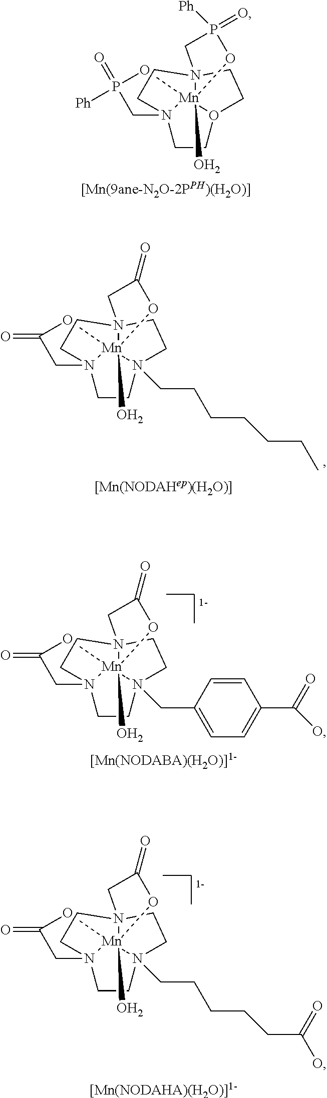

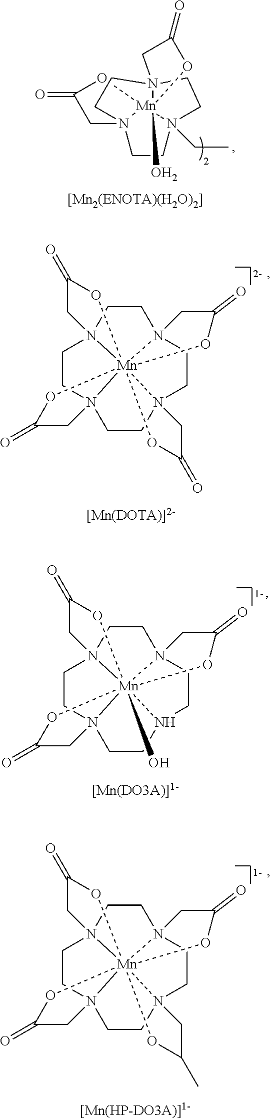

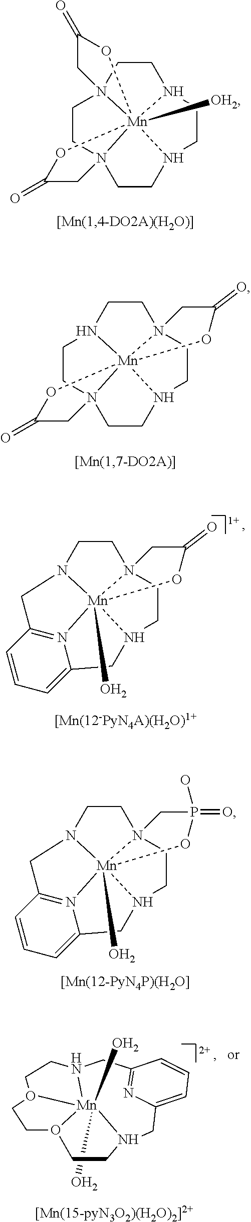

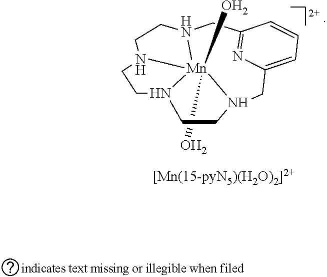

5. The contrast agent of claim 1, wherein the contrast agent has the chemical structure:

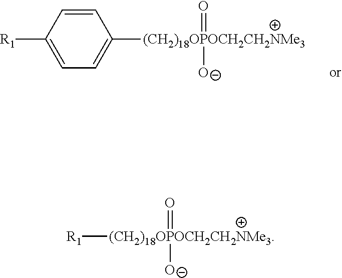

6. The contrast agent of claim 5, wherein the contrast agent, excluding the chelated Mn²⁺ isotope, has a chemical structure that is selected from the group consisting of:

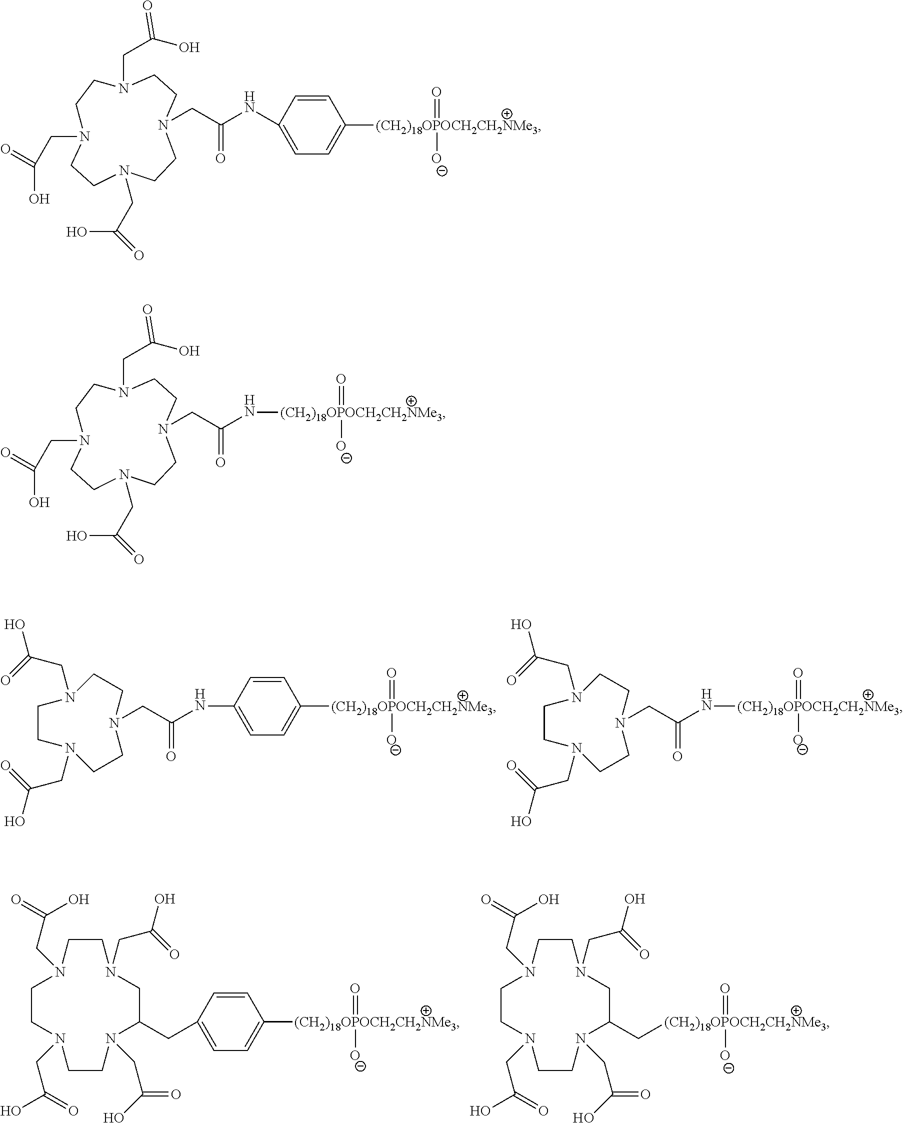

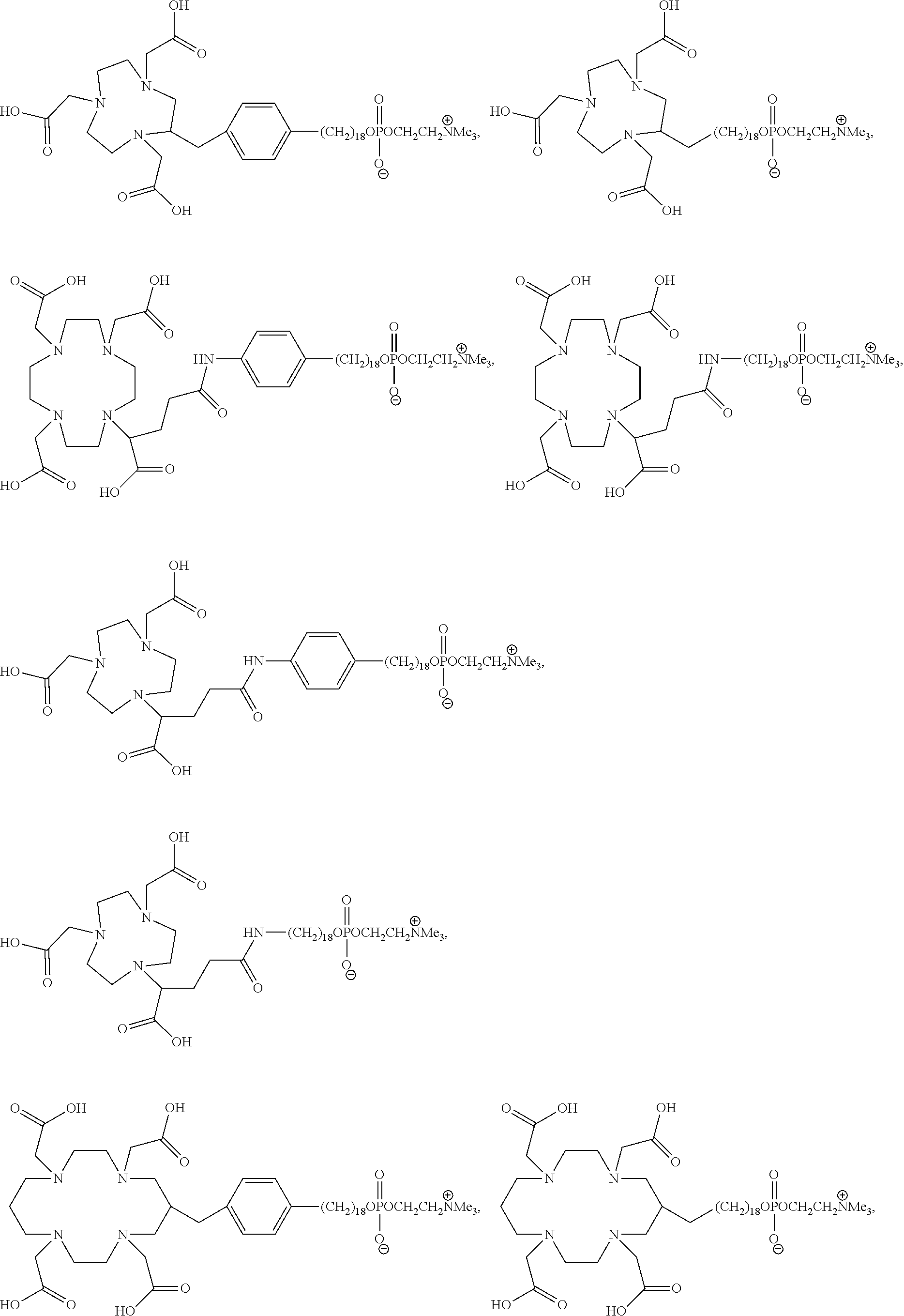

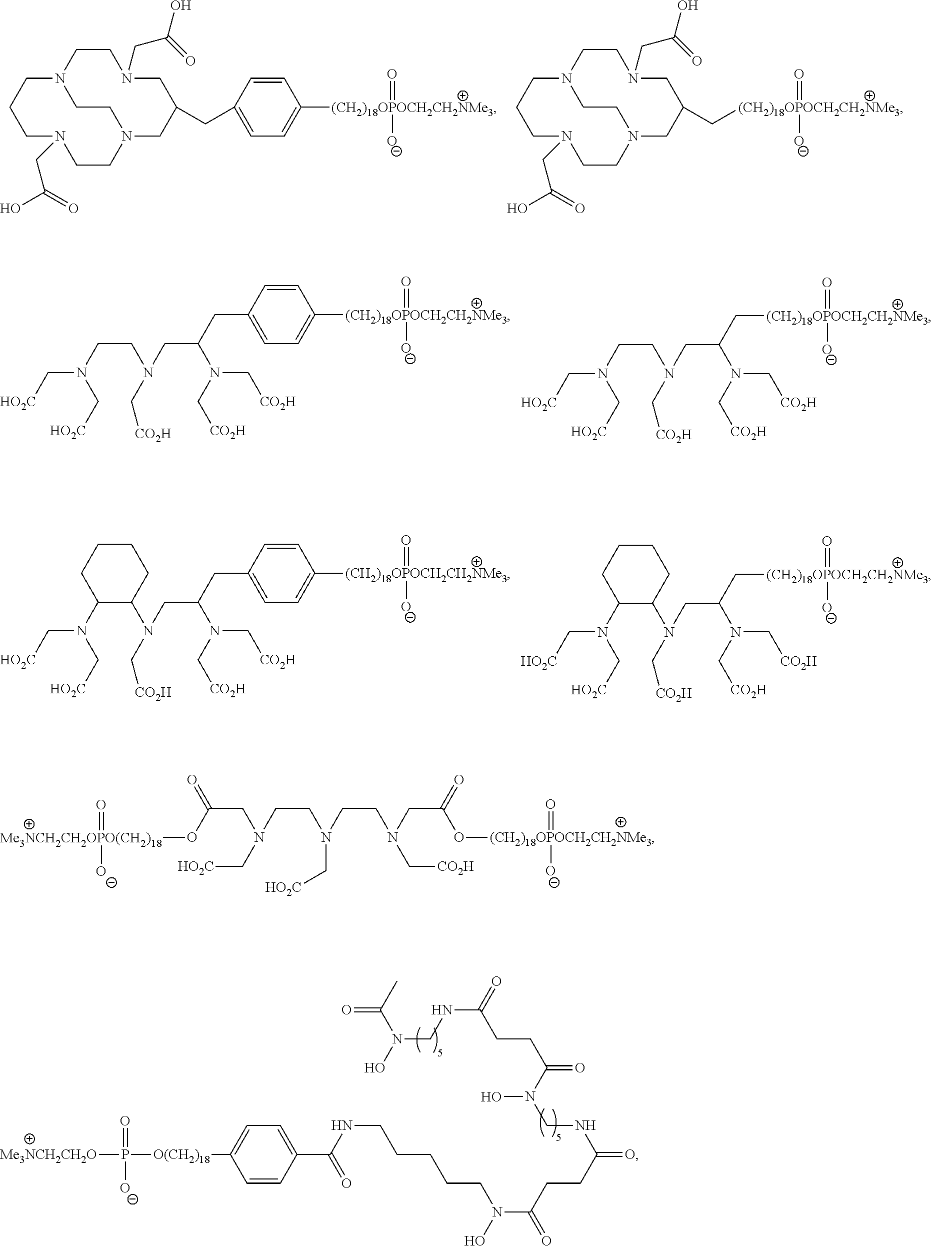

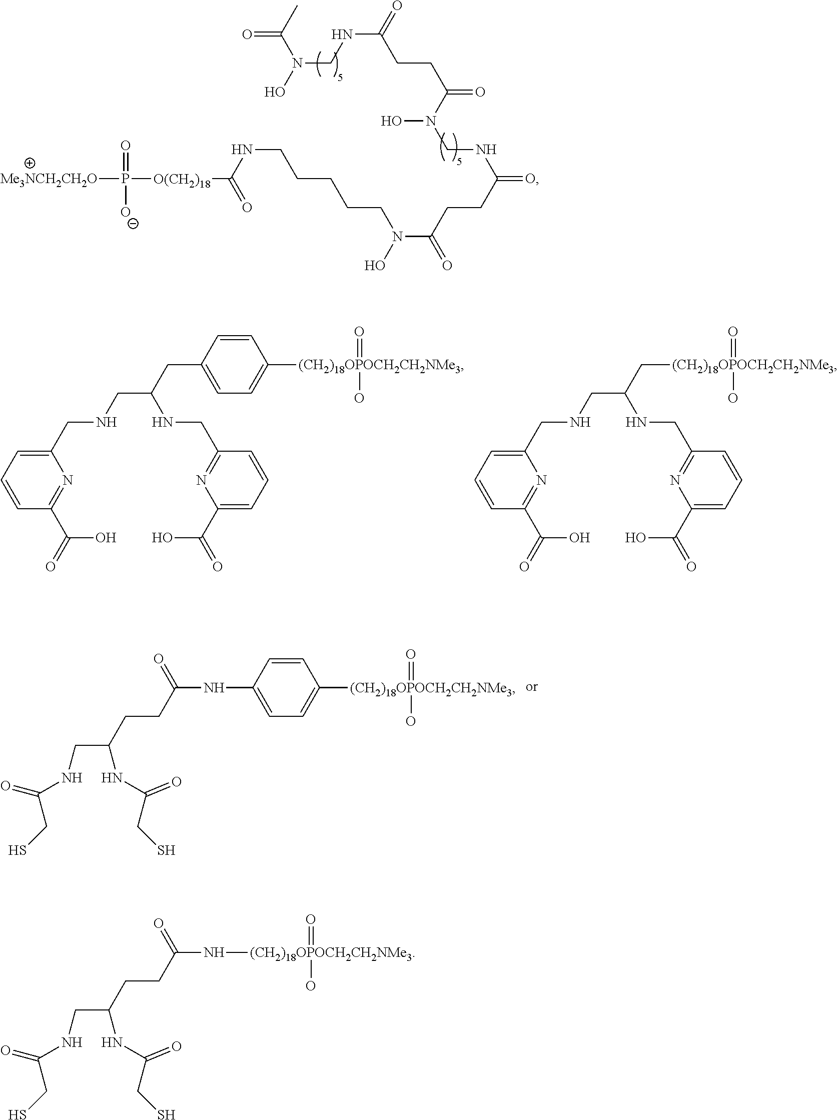

7. The contrast agent of claim 5, wherein the contrast agent has the chemical structure:

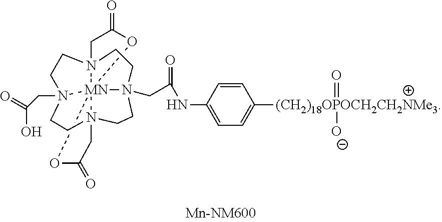

8. A composition comprising the contrast agent of claim 1 and a pharmaceutically acceptable carrier.

9. A method for detecting or imaging one or more cancer tumor cells in a biological sample, comprising:
(a) contacting the biological sample with the contrast agent of claim 1; and
(b) identifying individual cells or regions within the biological sample that are emitting signals characteristic of the chelated Mn²⁺ isotope, whereby one or more cancer tumor cells are detected or imaged.

10. The method of claim 9, wherein the step of identifying individual cells or regions within the biological sample that are emitting signals characteristic of the chelated Mn²⁺ isotope is performed using magnetic resonance imaging (MRI) or using both MRI and positron emission topography (PET) imaging.

11. The method of claim 9, wherein the biological sample is part or all of a subject.

12. The method of claim 11, wherein the contacting step is performed by injecting the contrast agent into the subject.

13. The method of claim 11, wherein the subject is a human.

14. The method of claim 9, wherein the step of identifying individual cells or regions within the biological sample that are emitting signals characteristic of the chelated Mn²⁺ isotope is performed using both MRI and PET, and wherein the contrast agent is the contrast agent that is used for both the MRI and PET.

15. The method of any of claim 9, wherein the cancer cells are adult solid tumor cells or pediatric solid tumor cells.

16. The method of claim 9, wherein the cancer cells are selected from the group consisting of melanoma cells, neuroblastoma cells, lung cancer cells, adrenal cancer cells, colon cancer cells, colorectal cancer cells, ovarian cancer cells, prostate cancer cells, liver cancer cells, subcutaneous cancer cells, squamous cell cancer cells, intestinal cancer cells, retinoblastoma cells, cervical cancer cells, glioma cells, breast cancer cells, pancreatic cancer cells, Ewings sarcoma cells, rhabdomyosarcoma cells, osteosarcoma cells, retinoblastoma cells, Wilms' tumor cells, and pediatric brain tumor cells.

17. A method of diagnosing cancer in a subject, comprising performing the method of claim 9, wherein the biological sample is obtained from, part of, or all of a subject, and whereby if cancer cells are detected or imaged, the subject is diagnosed with cancer.

18. The method of claim 17, wherein the cancer that is diagnosed is an adult solid tumor or a pediatric solid tumor.

19. The method of claim 18, wherein the cancer is selected from the group consisting of melanoma, neuroblastoma, lung cancer, adrenal cancer, colon cancer, colorectal cancer, ovarian cancer, prostate cancer, liver cancer, subcutaneous cancer, squamous cell cancer, intestinal cancer, retinoblastoma, cervical cancer, glioma, breast cancer, pancreatic cancer, Ewings sarcoma, rhabdomyosarcoma, osteosarcoma, retinoblastoma, Wilms' tumor, and pediatric brain tumors.

20. A method of monitoring the efficacy of a cancer therapy in a human subject, comprising performing the method of claim 9 at two or more different times on the biological sample, wherein the biological sample is obtained from, part of, or all of a subject, and whereby the change in strength of the signals characteristic of the Mn²⁺ isotope between the two or more different times is correlated with the efficacy of the cancer therapy.
